(19) United States
(12) Patent Application Publication
(10) Pub. No.: US 2006/0159742 A1
(43) Pub. Date: Jul. 20, 2006

(54) Stabilized individually coated ramipril particles, compositions and methods

(75) Inventors: Edward S. Wilson, Cary, NC (US); Kevin H. Sills, Apex, NC (US); M. King Jolly, Hillsborough, NC (US); Martin W. Beasley, Cary, NC (US); David P. Hause, Carrboro, NC (US)

Correspondence Address: JONES DAY, 222 EAST 41ST ST, NEW YORK, NY, 10017, US

(73) Assignee: King Pharmaceutical Research & Development, Inc.

(21) Appl. No.: 11/269,387

(22) Filed: Nov. 7, 2005

Related U.S. Application Data
(60) Provisional application No. 60/625,270, filed on Nov. 5, 2004.

(51) Int. Cl. A61K 31/401; A61K 9/48; A61K 9/20

(52) U.S. Cl. 424/451; 424/464; 514/423

(57) ABSTRACT

The present invention relates to novel ramipril crystalline particles with improved stability and bioavailability. More particularly, the present invention is directed to individually coated, single ramipril crystalline particles for pharmaceutical and biopharmaceutical applications in oral therapies that are stabilized against decomposition into degradation products, namely, ramipril-DKP and ramipril-diacid, during formulation and storage conditions. The present invention also relates to stabilized ramipril pharmaceutical compositions, novel anhydrous pharmaceutical grade ramipril powders, methods for improving ramipril bioavailability, and methods of manufacture and stabilization of ramipril formulations. The novel, anhydrous pharmaceutical grade ramipril powders and ramipril compositions and dosage forms formed therewith are useful in the treatment of cardiovascular disorders and have the advantage that they provide greater stability against decomposition into ramipril-DKPs and ramipril-diacids under formulation and storage conditions. In addition, they maintain consistent label ramipril potency over extended shelf-life and provide reduced in vivo variability in the bioavailability of ramipril among subjects when administered orally.

This application claims the benefit of U.S. Provisional Application No. 60/625,270, filed Nov. 5, 2004 the contents of which are incorporated herein in its entirety.

### FIELD OF THE INVENTION

The present invention relates to novel ramipril particles with improved stability and bioavailability. More particularly, the present invention is directed to individually coated, single ramipril particles for biopharmaceutical applications in oral therapies that are stabilized against decomposition into degradation products, namely, ramipril-DKP and ramipril-diacid. Such ramipril particles of the present invention are capable of withstanding formulation and storage conditions that can cause degradation or decomposition. The present invention also relates to stabilized ramipril pharmaceutical compositions, methods for improving ramipril bioavailability, and methods of manufacture and stabilization of ramipril formulations.

### BACKGROUND

Today, over 50 million Americans suffer from cardiovascular disease. It is believed to be the number one cause of death and disability in the United States. In fact, more women in the United States die of heart disease than of all cancers combined.

Because cardiovascular disease generally progresses silently in the early stages, detection and diagnosis is difficult. Consequently, cardiovascular disease is frequently under-diagnosed and under-treated. Therefore, by the time that cardiovascular disease is detected or diagnosed, the disease is usually quite advanced, sometimes too advanced to permit successful treatment or prevention of serious disability or death.

Cardiovascular disease includes, but is not limited to, arterial enlargement, arterial narrowing, peripheral artery disease, atherosclerotic cardiovascular disease, high blood pressure, angina, irregular heart rates, inappropriate rapid heart rate, inappropriate slow heart rate, angina pectoris, heart attack, myocardial infarction, transient ischemic attacks, heart enlargement, heart failure, congested heart failure, heart muscle weakness, inflammation of the heart muscle, overall heart pumping weakness, heart valve leaks, heart valve stenosis (failure-to-open fully), infection of the heart valve leaflets, heart stoppage, asymptomatic left ventricular dysfunction, cerebrovascular incidents, strokes, chronic renal insufficiency, and diabetic or hypertensive nephropathy.

Angiotensin II is a very potent vasoconstrictor chemical that is responsible for controlling blood pressure in humans. Angiotensin II controls blood pressure by causing the muscles surrounding the blood vessels in the body to narrow or constrict. When the blood vessels are narrowed, the pressure within the constricted blood vessels increases making it much more difficult for the heart to pump blood through them. Unfortunately, it is this increase in vascular resistance that can lead to high blood pressure (hypertension) in people.

Angiotensin II is formed in the blood and tissue from Angiotensin I. The conversion of Angiotensin I into Angiotensin II is catalyzed by a peptidyl dipeptidase enzyme known as the angiotensin converting enzyme (ACE). By blocking the ACE enzyme and the formation of Angioensin II, blood vessel constriction and pressure can be controlled. As a result, the blood vessels enlarge or dilate, and the blood pressure is reduced. This lower blood pressure makes it easier for the heart to pump blood. This action will reduce oxygen consumption by the heart, thereby improving cardiac output or heart function and moderate left ventricular and vascular hypertrophy. In addition, the progression of kidney disease due to high blood pressure or diabetes may be slowed.

ACE Inhibitors (angiotensin-converting enzyme inhibitors) are encompassed in a class of drugs that were first introduced in about 1981. ACE inhibitors work by blocking the action of the ACE enzyme in human subjects and animals. The ACE inhibitors accomplish this blocking action by binding to the zinc component of the ACE enzyme. While ACE inhibitors are pharmacologically similar, they differ from one another, for example, in chemical structure, how they are eliminated from the body and their doses. Some ACE inhibitors need to be converted into an active form in the body before they work. In addition, some ACE inhibitors may work more on the ACE enzyme that is found in tissues than on the ACE enzyme that is present in the blood.

In view of these differences, ACE inhibitors can be divided into three subgroups: sulfhydryl-containing ACE inhibitors exemplified by captopril; carboxyl or dicarboxyl-containing ACE inhibitors, such as enalapril and ramipril; and phosphorous or phosphinyl ACE inhibitors, such as fosinopril. There are several ACE inhibitors currently on the market. The following is a list of the ACE inhibitors that are available in the United States: captopril (Capoten®), benazepril (Lotensin®), enalapril (Vasotec®), lisinopril (Prinivil®, Zestril®), fosinopril (Monopril®), ramipril (Altace®), perindopril (Aceon®), quinapril (Accupril®), moexipril (Univasc®), and trandolapril (Mavik®).

When first introduced in 1981, ACE inhibitors were used only to treat hypertension. Today ACE inhibitors are commonly used for controlling blood pressure and treating congestive heart failure, myocardial infarction, diabetes mellitus, chronic renal insufficiency and atherosclerotic cardiovascular disease, and preventing kidney damage in people with hypertension or diabetes. It has been shown in certain studies that individuals with hypertension, heart failure or prior heart attacks, who were treated with an ACE inhibitor, lived longer than patients who did not take an ACE inhibitor (Though out this application patient and subject can be used interchangeably). Clinical outcomes of ACE inhibition include decreases in myocardial infarction (fatal and nonfatal), reinfarction, angina, stroke, end-stage renal disease, and morbidity and mortality associated with heart failure. ACE inhibitors are generally well tolerated and have few contraindications. See, for example, Am. Fam. Physician, 66:461-8, 473 (2002). Because ACE inhibitors may prevent early death resulting from hypertension, heart failure or heart attacks, ACE inhibitors are believed to be one of the most important groups of drugs on the market today.

Ramipril is an important ACE inhibitor used in the treatment of cardiovascular disease, especially hypertension and nephropathia, and it is one of the most frequently prescribed drugs for congestive heart failure. In hypertensive patients, ramipril is known to cause a reduction in peripheral arterial resistance, and thus, a reduction in blood pressure without a compensatory rise in heart rate. Ramipril has also been shown to reduce mortality in patients with clinical signs of congestive heart failure after surviving an acute myocardial infarction. Ramipril has been suggested to have an added advantage over many other ACE inhibitors due to its pronounced inhibition of the ACE enzymes in tissues resulting in organ protective effects, e.g., in the heart, kidney, and blood vessels.

Ramipril is an ethyl ester. It is a prodrug and a long-acting ACE inhibitor. Its active metabolite is ramiprilat, which is obtained in vivo upon administration of ramipril. Ramipril is converted to ramiprilat in the body by hepatic cleavage of the ester group. Ramiprilat, the diacid or free acid metabolite of ramipril, is a non-sulfhydryl angiotensin converting enzyme inhibitor.

Ramipril, a 2-aza-bicyclo [3.3.0]-octane-3-carboxylic acid derivative, is a white, crystalline particular substance or powder that is soluble in polar organic solvents and buffered aqueous solutions. The ramipril crystalline particles are columnar (or needle like) in shape. The ramipril crystalline particles melt between about 105° C. and about 112° C. Ramipril and processes for making and using ramipril are described and claimed in U.S. Pat. Nos. 4,587,258, 5,061,722 and 5,403,856, all of which are incorporated herein by reference in their entirety. The preparation of ramipril has also been described in EP 0 079 022 A2, EP 0 317 878 A1 and DE 44 20 102 A, which are incorporated herein by reference in their entirety.

The CAS Registry Number for ramipril ethyl ester is 87333-19-5. The manufacturer's code is HOE 498, S81 3498, Delix®. Minimum purity for ramipril is 980 g/kg. Ramipril's chemical or IUPAC name is (2S,3aS,6aS)-1[(S)-N-[(S)-1-Carboxy-3-phenylpropyl]alanyl]octahydrocyclopenta[b]pyrrole-2-carboxylic acid, 1-ethyl ester. Its empiric formula is C₂₃H₃₂N₂O₅, and its molecular weight is 416.5. The chemical structure for ramipril ethyl ester is:

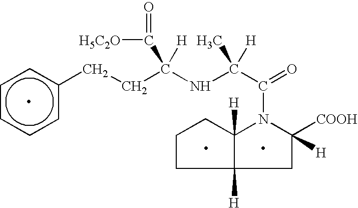

Ramipril ethyl ester is marketed in the United States under the brand name Altace® and abroad under the brand name Delix®.

Altace® (ramipril) is supplied as hard shell capsules for oral administration containing 1.25 mg, 2.5 mg, 5 mg or 10 mg of ramipril. The inactive ingredients present are pregelatinized starch NF, gelatin, and titanium dioxide. The 1.25 mg capsule shell contains yellow iron oxide, the 2.5 mg capsule shell contains D&C yellow #10 and FD&C red #40, the 5 mg capsule shell contains FD&C blue #1 and FD&C red #40, and the 10 mg capsule shell contains FD&C blue #1.

Even though ramipril is without question one of the most important ACE inhibitors available today, ramipril can be unstable in some pharmaceutical formulations. According to EP 0317878 A1, U.S. Pat. Nos. 5,442,008 and 5,151,433, PCT/EP2004/000456 and PCT/CA02/01379, this instability can be influenced by several factors, such as mechanical stress, compression, manufacturing processes, excipients, storage conditions, heat and moisture. Consequently, ramipril needs special care when formulating into pharmaceutical preparations to minimize the decomposition of ramipril into degradation products.

The degradation of ramipril is believed to occur mainly via two pathways: (a) hydrolysis to ramipril-diacid; and (b) cyclization or condensation to ramipril-diketopiperazine (ramipril-DKP), as described in U.S. Pat. Nos. 5,442,008 and 5,151,433 and PCT/EP2004/00456.

Various attempts have been made to stabilize ramipril in pharmaceutical formulations. PCT/EP2004/00456 describes a process to formulate ramipril compositions that utilizes excipients with low water content and processing parameters and packaging material that prohibit water or moisture uptake. PCT/EP2004/00456 does not teach ramipril formulations comprising individually coated, stabilized ramipril particles. Moreover, the ramipril compositions described in PCT/EP2004/00456, have a high rate of ramipril-DKP formation of 9.56% after two months at ambient temperature and humidity. Additionally, even when placed in air-tight packaging, the ramipril compositions have a rate of ramipril-DKP formation of 2.0%, after one month at 40° C. and at 75% humidity.

PCT/CA2002/01379 describes solid ramipril capsules that comprise a mixture of ramipril and lactose monohydrate as the diluent. According to PCT/EP2004/000456, the process includes lactose monohydrate as the major excipient to formulate ramipril compositions in an attempt to improve ramipril stability. However, PCT/CA2002/01379 does not teach ramipril formulations comprising individually coated, stabilized ramipril particles and immediately after formation of the described capsules, ramipril-DKP formation is already at 1.10%.

U.S. Pat. Nos. 5,442,008 and 5,151,433 describe yet another attempt to overcome instability by reporting the use of a polymeric protective coating. According to U.S. Pat. Nos. 5,442,008 and 5,151,433, an active substance is dispersed with a solution or dispersion of a film-former in a suitable kneader, mixer or mixer-granulator to form a uniformly wetted composition that is then forced through a screen and dried into granules. The dried granules formed are passed again through a screen and then used to manufacture capsules or tablets. A coating may be obtained in a fluidized bed. The particles of active substance are sprayed in the stream of air with a solution or dispersion of the polymer and are dried. The coated granules of active substance can be used immediately after the drying process for filling capsules or for manufacturing tablets. It is also possible to combine the two processes together by initially wetting the active substance with the solution or dispersion of a polymer in a kneader, mixer or mixer-granulator, and subsequently processing it by granulation to give homogeneous agglomerates that are then finally coated with the solution or dispersion of the polymer in a fluidized bed. The resulting ramipril agglomerates have many various disadvantages.

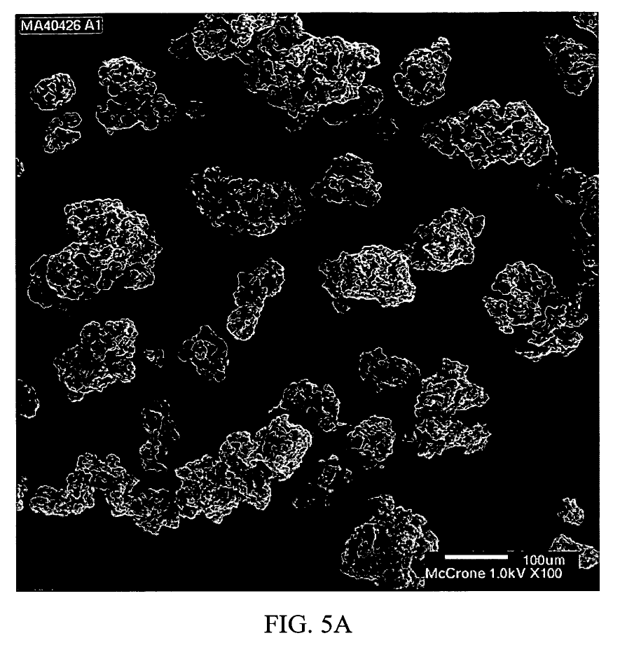

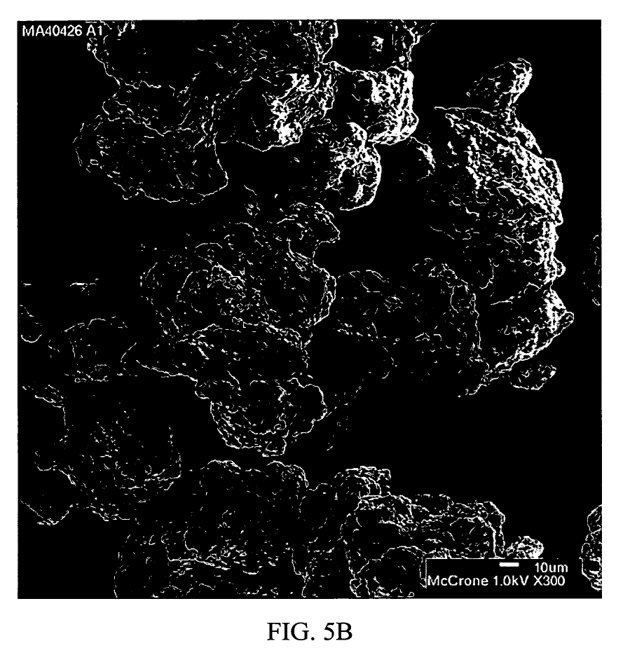

One example of such ramipril agglomerates is the GeCoated ramipril agglomerate, manufactured by Aventis Pharma Deutschland GmbH (Frankfurt on Main, Germany). GeCoated ramipril agglomerates are ramipril agglomerates coated with a hydroxypropyl methylcellulose polymer coating (1.192 mg GeCoated granules=1.0 mg ramipril). Unfortunately, these GeCoated agglomerates, which rely on the polymer coating for stabilization, may have ramipril particles or portions of ramipril particles that remain uncoated and, thus, are unprotected. FIGS. 5A, 5B and 5C show portions of exposed ramipril in GeCoated ramipril agglomerates that is susceptible to degradation to ramipril-DKP or ramipril-diacid during formulation and storage. GeCoated agglomerates also have the disadvantage of becoming de-agglomerated (broken apart) during processing. As agglomerated particles are separated (broken apart), uncoated ramipril is exposed and becomes unprotected against manufacturing stresses and environmental conditions making the exposed ramipril prone to the degradation the coating was originally intended to prevent.

Additionally, shear forces are unavoidable, especially when manufacturing solid oral dosage forms. High-shear forces are usually desired to achieve content uniformity of low dose solid oral products. The use of high-shear blenders, intensifier-bars, choppers and milling equipment are common in the pharmaceutical industry when manufacturing these types of products. As such, the need to avoid the creation and use of agglomerates when preparing a stabilized material is of importance for the viability of such processes that require high-shear forces.

Another disadvantage associated with agglomerates concerns the process of agglomeration (sticking individual particles together) itself, which may change the particle size distribution of the powder from that of the original material. The overall particle size of the coated agglomerated product generally ends up larger then that of the original material and the surface area is thus significantly reduced. Due to the trend in the pharmaceutical industry to move toward low dose drugs and dry blend, direct compression formulations, controlling particle size and surface area are critical to one's ability to create a cost effective, uniform, high quality product.

As such, despite past attempts to stabilize ramipril compositions, there still remains a need to develop ramipril compositions that have significantly improved stability, i.e., that resist or prevent the degradation of ramipril to ramipril-DKP and ramipril-diacid, its major decomposition products, under formulation and storage conditions, so that label potency remains more consistent over the shelf-life of such ramipril compositions.

Citation of any reference in the Background section of this application is not an admission that the reference is prior art to the application.

### SUMMARY

In brief, the present invention alleviates and overcomes problems and shortcomings relating to ramipril instability through the discovery that novel ramipril crystalline particles can improve stability and maintain potency of ramipril in solid oral dosage forms under formulation and extended shelf-life conditions.

The present invention therefore is directed to novel ramipril particles that are substantially stable against decomposition into degradant products, such as ramipril-diacid and ramipril-DKP (ramipril-DKP), novel anhydrous, pharmaceutical grade ramipril powders, novel stabilized ramipril pharmaceutical compositions having improved bioavailability, novel methods for improving ramipril bioavailability, and methods of manufacture and stabilization of ramipril formulations.

It has now been discovered that stable ramipril formulations can be accomplished by coating single ramipril API crystalline particles individually with a suitable coat forming material prior to formulation or being compressed into solid oral ramipril dosage forms. In other words, it has now been discovered that, when each ramipril crystalline particle is individually and effectively coated and protected with a coat forming material, ramipril stability and potency consistency can be quite unexpectedly improved and maintained through formulation processing and over an extended shelf-life of the drug product.

Thus, solid oral ramipril pharmaceutical compositions formulated with discrete or stand alone individually coated ramipril crystalline particles in accordance with the present invention are improved over prior solid oral ramipril compositions, because such novel compositions will retain a higher percentage of their potency over a longer period of time than the same compositions formulated with ramipril crystalline particles that have not been individually coated or stabilized.

In accordance with the present invention, the novel stabilized ramipril crystalline particles of the present invention are individually and sufficiently coated or surrounded with a suitable coat forming material so that no portion of a single ramipril crystalline particle remains unprotected or exposed to the atmosphere or the environment before, during or after formulation and during storage. It has been discovered that the use of such individually coated, single ramipril crystalline particles in the compositions of the present invention substantially increases the stability and maintains the potency of ramipril, so that the patients now treated with ramipril will obtain more consistent potency and bioavailability over the extended drug product shelf-life, especially when compared to prior ramipril drug products available heretofore.

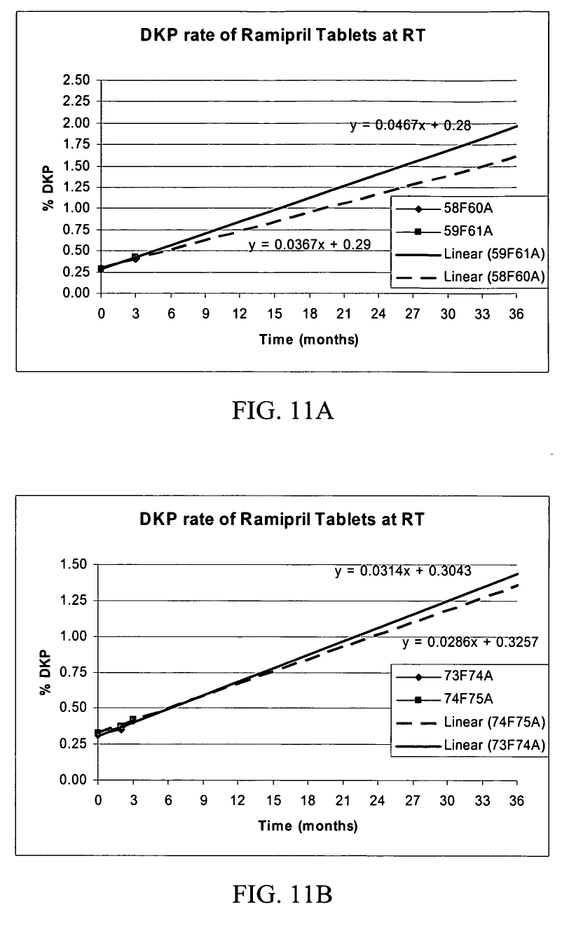

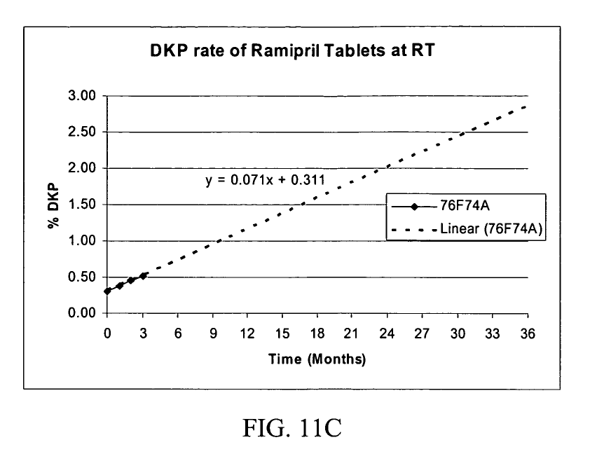

By way of example, it is surprisingly found that, when individual ramipril crystalline particles are coated and stabilized in accordance with the present invention, the formation of ramipril-DKP in compositions employing such stable, individually coated, single ramipril particles over the shelf-life of such compositions is less than about 0.3% during about the first three months and less than about 3.0% during a period of at least about 36 months from the date that the such compositions are first formulated. Preferred individually coated ramipril particles have ramipril-DKP formation of less than about 0.3% during about the first three months and less than about 2.0% during such extended period, and more preferred individually coated ramipril particles have ramipril-DKP formation of less than about 0.3% during about the first three months and less than about 1.5% during such extended period. See FIGS. 11A, 11B and 11C. It has been found that this result is an unexpected and significant improvement, especially when compared to the stability or loss of potency of the ramipril compositions stored under the same conditions, but formulated with uncoated ramipril crystalline particles.

Thus, the stabilized, individually coated, single ramipril crystalline particles of the present invention provide the basis for novel stabilized ramipril compositions that have remarkably improved stability and biopharmaceutical profiles and are particularly advantageous for oral delivery.

In accordance with a further aspect of the present invention, the novel stabilized, individually coated, single ramipril crystalline particles may be formulated with any suitable pharmaceutically acceptable excipients and formed into any solid dosage forms, such as capsules, caplets, tablets, tablet-filled capsules, puvules, granules, powders or the like, for oral administration, using any suitable compounded or formulation techniques.

Particularly advantageous aspects of the present invention include the stable, stand alone, individually coated ramipril crystalline particles formulated into tablet form, which has significantly improved stability and shelf-life. Tablets or other solid oral dosage forms, as contemplated by the present invention, may be in any effective ramipril amount, e.g., 1.25, 2.5, 5.0, 7.5, 10, 12.5, 15, 20, 25, 30, 40, 50, 60, 70, 75, 80, 90, 100 mg or higher. When oral tablet dosage forms are selected, the tablets may be of any suitable size and shape, such as round, square, rectangular, oval, diamond, pentagon, hexagon, or triangular shapes. Of particular interest are tablets and capsules, including tablet-filled capsules; especially of interest are 15 mg ramipril tablets, 15 mg ramipril caplets, 15 mg ramipril capsules and 15 mg ramipril tablet-filled capsules.

In accordance with the present invention, solid crystalline ramipril API particles, as obtained from the Aventis Pharma Deutschland GmbH (Frankfurt on Main, Germany), are preferred as the starting ramipril crystalline particles to be coated with a coat forming material in accordance with the present invention. Other suitable sources of ramipril include, but are not limited to, Brantford Chemicals, Molcan Corporation or Bio-Gen Extracts.

Nevertheless, in some applications, it may be desirable to prepare ramipril crystalline particles in accordance with U.S. Pat. Nos. 5,061,722 and 5,403,856, or to prepare micro- or nanoparticles, as such preparations may provide for more rapid bioavailability when orally administered.

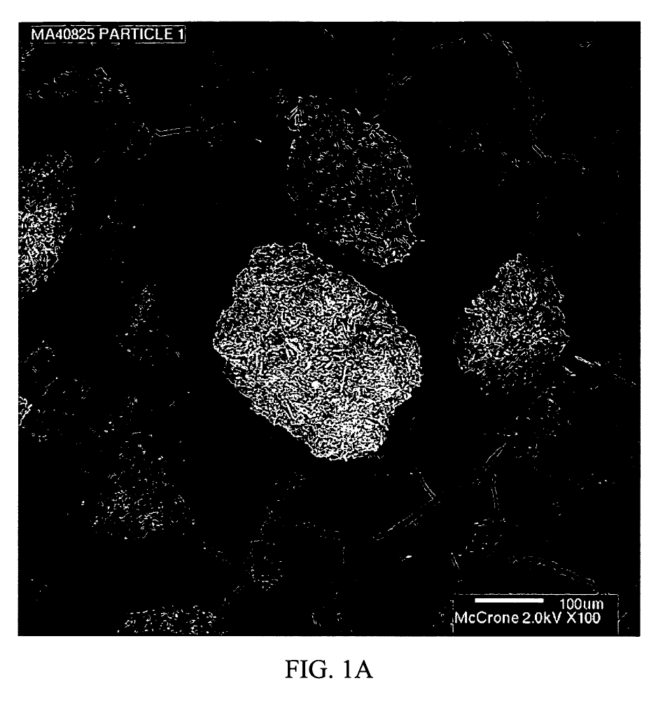

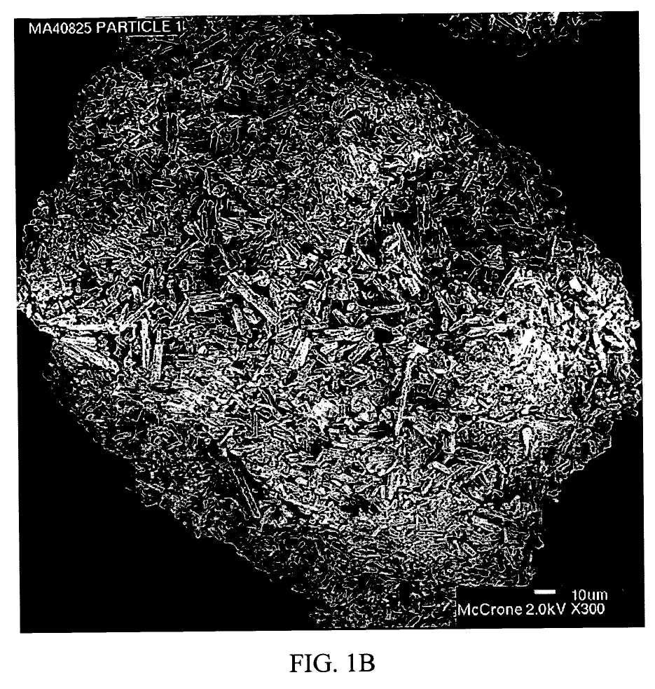

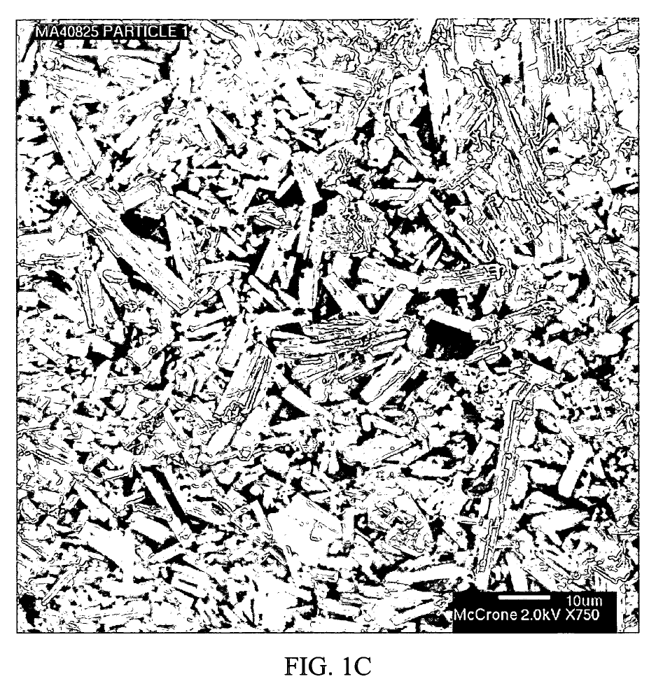

In a further aspect of the present invention, a process that effectively coats or encapsulates the surface of each single ramipril crystalline particle with a pharmaceutically acceptable stabilizing coat forming material, regardless of the physical form or shape of the ramipril particles, is contemplated. As illustrated by the SEM images shown in FIGS. 1, 2 and 3, the individually coated, single ramipril crystalline particles of the present invention are completely coated or surrounded with a coat forming material.

In accordance with this aspect of the invention, a coating process is employed to preferably completely and uniformly coat each individual ramipril particle with a coat forming material. Generally speaking, the coating process, in accordance with the present invention, comprises suspending or dispersing single ramipril crystalline particles in a liquid phase, into which a coat forming material has been dissolved; coating the single ramipril particles; removing the water or drying the liquid phase to precipitate discrete, individually coated, ramipril particles from the liquid phase; and collecting the precipitated, individually coated, single ramipril particles to form a novel, anhydrous pharmaceutical grade ramipril powder.

In accordance with this aspect of the invention, a spray-drying process is preferably employed. In this procedure, single crystalline particles of ramipril are first suspended in a liquid phase comprising a coat forming material to form dispersion. The dispersion is then spray-dried to form the novel stabilized individually coated, single ramipril crystalline particles of the present invention. Control of particle size and spray-drying conditions are believed to be important because it is necessary for the entire surface of each ramipril particle to be capable of protecting the ramipril particles from the atmosphere and degradation into ramipril-DKP and ramipril-diacid under formulation and storage conditions. Preferably, each particle is completely covered, or substantially completely covered.

Overall and in general, the invention encompasses solid pharmaceutical compositions comprising stabilized, individually coated, single ramipril crystalline particles, where the coating protects the single ramipril particles from degradation, yet allows appropriate release of the ramipril, i.e., does not interfere with the bioavailability over the life of such compositions. Thus, the disclosed ramipril preparations formulated with stabilized, individually coated, single, ramipril crystalline particles differ from previously prepared ramipril preparations that have surface area exposure of the active ramipril crystalline particles, due in part to the fact that the individual ramipril crystalline particles are not completely or substantially completely coated. Because oral solid ramipril dosage forms in the past have not been prepared with individually coated, single ramipril crystalline particles, they have had problems associated with stability, loss of label potency and ramipril-DKP production.

The stabilized, individually coated, single ramipril particles and the solid oral ramipril pharmaceutical compositions of the present invention are useful to prevent and/or treat cardiovascular disorders, such as hypertension, heart failure, congestive heart failure, myocardial infarction, atherosclerotic cardiovascular disease, asymptomatic left ventricular dysfunction, chronic renal insufficiency, and diabetic or hypertensive nephropathy.

In a further aspect of the invention, solid oral ramipril pharmaceutical compositions are formulated with the stabilized, individually coated ramipril particles of the present invention. More specifically, it has been surprisingly found that, when ramipril drug products are formulated with the stabilized, individually coated, single ramipril crystalline particles in accordance with the present invention, shelf-life can be extended to at least about 36 months without adversely affecting potency consistency, i.e. a loss of potency due to DKP formation over the shelf-life of the ramipril product is less than about 0.09% potency per month on average. In other words, ramipril pharmaceutical compositions of the present invention are stabilized for at least about 36 months from the date that the ramipril pharmaceutical compositions are first formulated. It has been found that this result is an unexpected and significant improvement, especially when compared to the stability or loss of potency of ramipril compositions stored under the same conditions, but formulated with uncoated ramipril particles.

Thus, an object of the present invention is to provide novel stabilized ramipril particles for formulating into solid oral dosage forms to increase stability over extended shelf-life of the ramipril pharmaceutical compositions.

It is another object of the present invention to produce novel stabilized, individually coated, single particles of ramipril that retain particle characteristics necessary to manufacture an acceptable uniform low dose, dry blend, and/or direct compression product as a solid oral dosage form.

It is another object of the present invention to produce a novel stabilized, anhydrous, pharmaceutical grade ramipril particle.

It is still another object of the present invention to produce a novel ramipril pharmaceutical grade powder, consisting essentially of unagglomerated, stabilized, anhydrous, individually coated, single ramipril crystalline particles, that is suitable for formulation into pharmaceutical dosage forms.

Another object of the present invention is to provide novel stabilized ramipril pharmaceutical compositions that have increased stability during formulation and over extended shelf-life and improved bioavailability.

Another object of the present invention is to provide methods to coat the individual ramipril particles with a coat forming material to stabilize, individual ramipril crystalline particles and for formulating solid oral dosage forms that have remarkably improved stability, potency and biopharmaceutical profiles over extended shelf-life.

Another object of this invention is to provide information to prescribing physicians and patients receiving ramipril therapy useful in maximizing the therapeutic effect of the oral dosage form.

Still another aspect of this invention is an article of manufacture that comprises a container containing a pharmaceutical composition comprising the coated ramipril particles in accordance with the present invention wherein the container holds preferably the ramipril composition in unit dosage form and is associated with printed labeling instructions advising of the stability, bioavailabilty and label potency.

These and other objects, features, and advantages of the present invention may be better understood and appreciated from the following detailed description of the embodiments thereof, selected for purposes of illustration and shown in the accompanying figures and examples. It should therefore be understood that the particular embodiments illustrating the present invention are exemplary only and not to be regarded as limitations of the present invention.

### BRIEF DESCRIPTION

FIG. 1A is spray-dried ramipril, 10% solids/5% coating at 100-fold magnification.

FIG. 1B is spray-dried ramipril, 10% solids/5% coating at 300-fold magnification.

FIG. 1C is spray-dried ramipril, 10% solids/5% coating at 750-fold magnification.

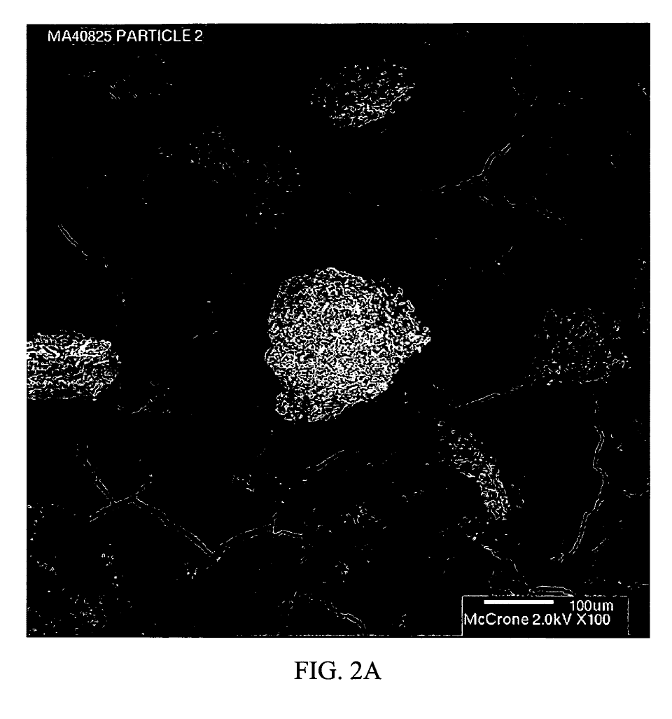

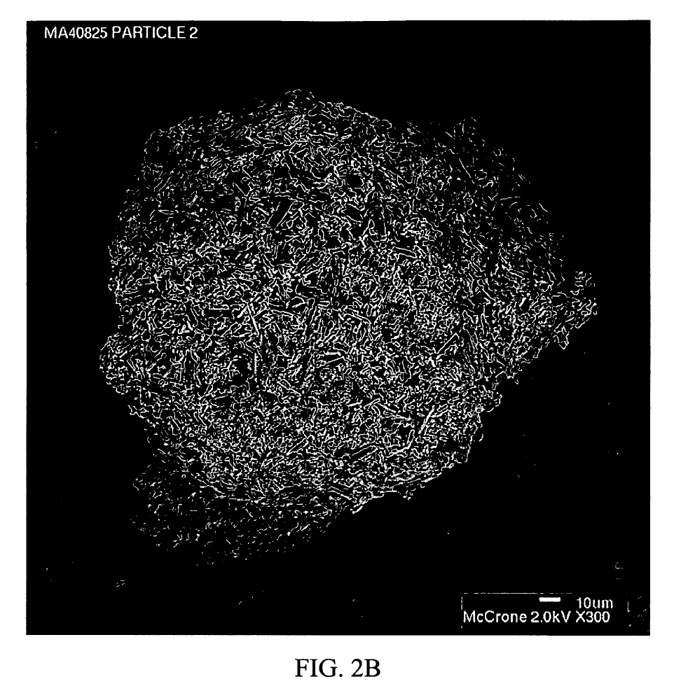

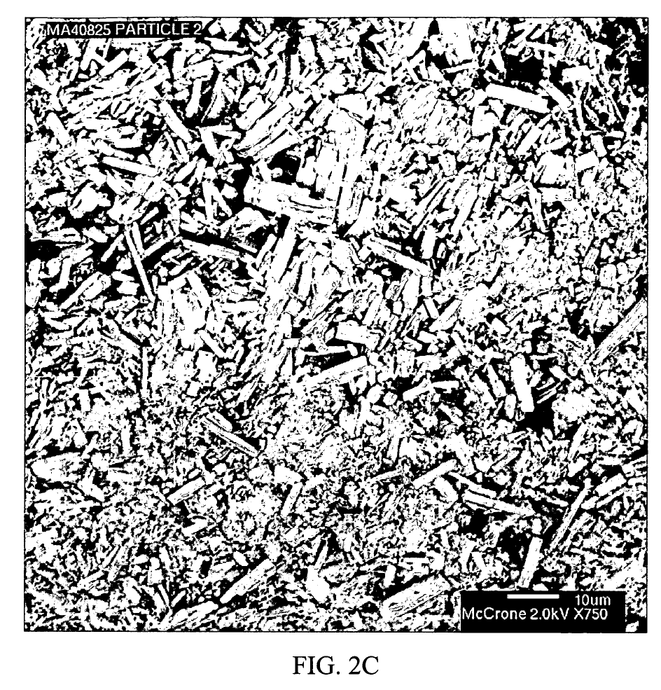

FIG. 2A is spray-dried ramipril, 10% solids/5% coating at 100-fold magnification.

FIG. 2B is spray-dried ramipril, 10% solids/5% coating at 300-fold magnification.

FIG. 2C is spray-dried ramipril, 10% solids/5% coating at 750-fold magnification.

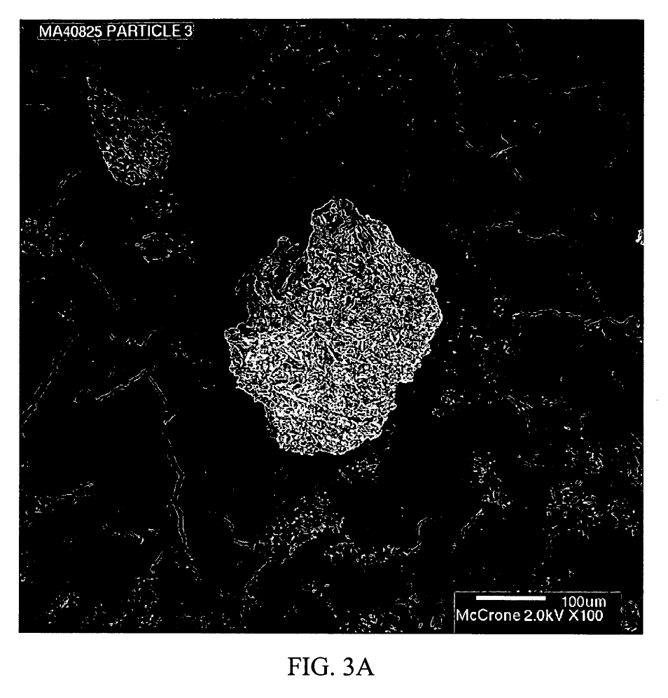

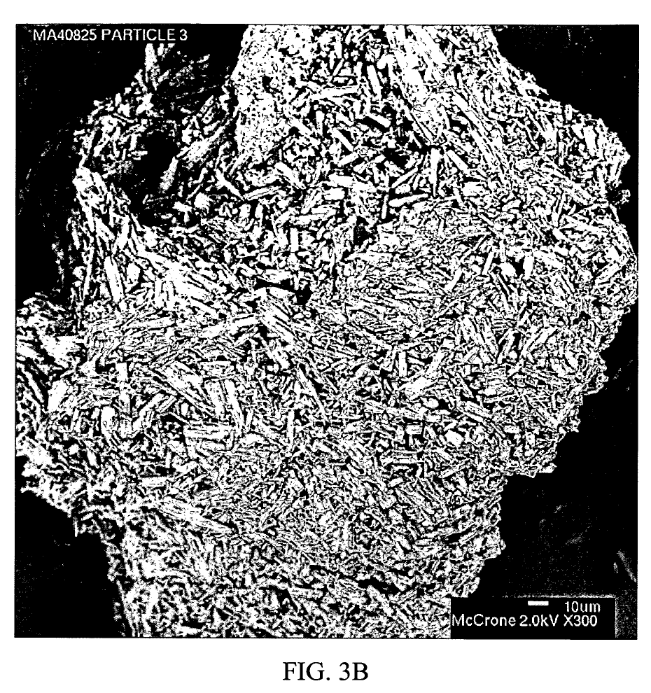

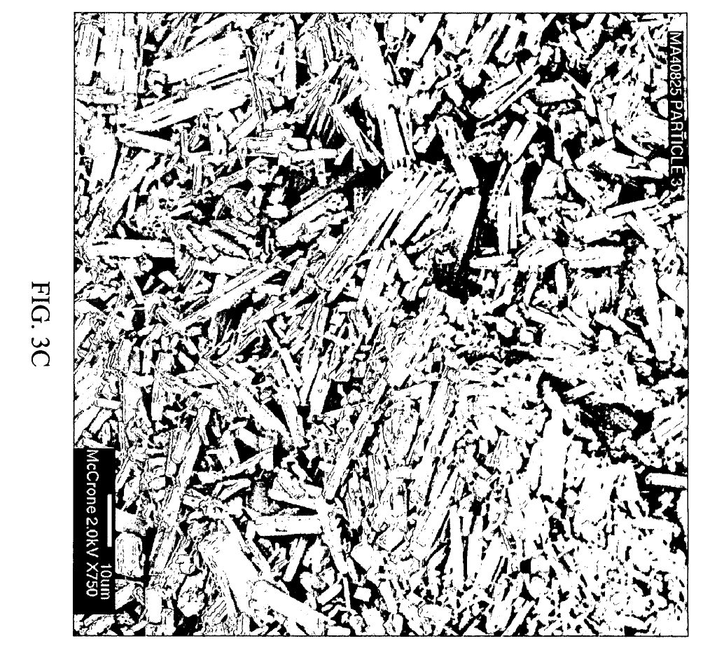

FIG. 3A is spray-dried ramipril, 10% solids/5% coating at 100-fold magnification.

FIG. 3B is spray-dried ramipril, 10% solids/5% coating at 300-fold magnification.

FIG. 3C is spray-dried ramipril, 10% solids/5% coating at 750-fold magnification.

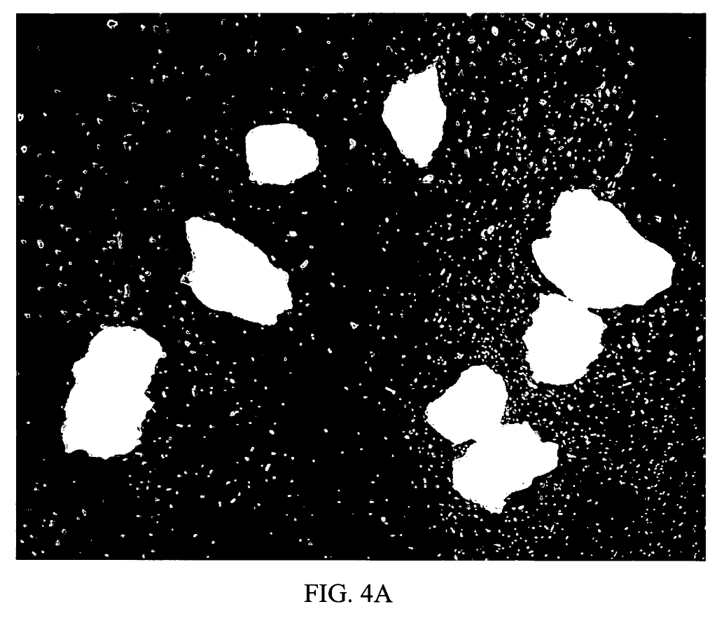

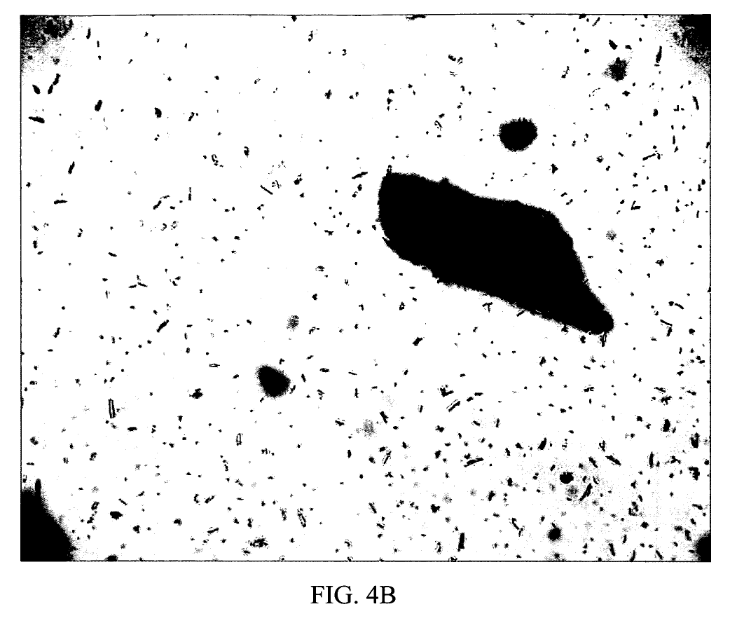

FIG. 4A shows large crystal agglomerates in ramipril spray-dried solids, coating wet at 40-fold magnification using reflected light.

FIG. 4B shows large crystal agglomerates in ramipril spray-dried solids, coating wet at 100-fold magnification using reflected light.

FIG. 5A is an electron micrograph of GeCoated Ramipril API screened to 425 μm through #40 mesh at 100-fold magnification.

FIG. 5B is an electron micrograph of GeCoated Ramipril API screened to 425 μm through #40 mesh at 300-fold magnification.

FIG. 5C is an electron micrograph of GeCoated Ramipril API screened to 425 μm through #40 mesh at 750-fold magnification.

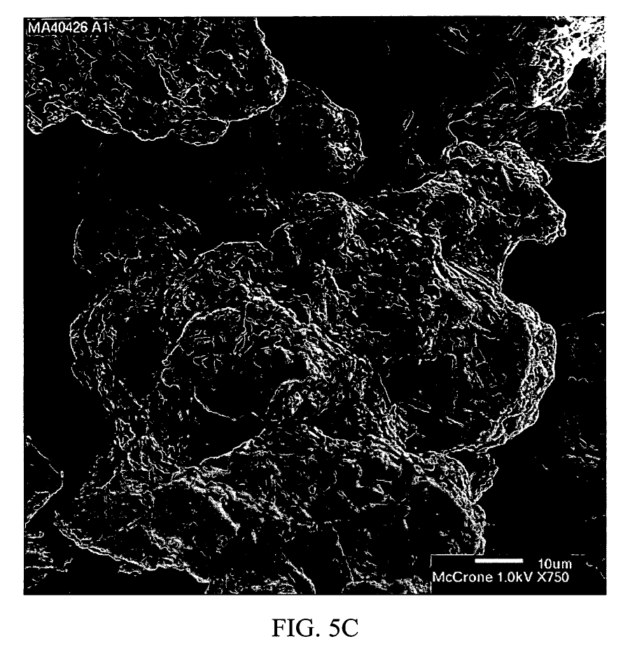

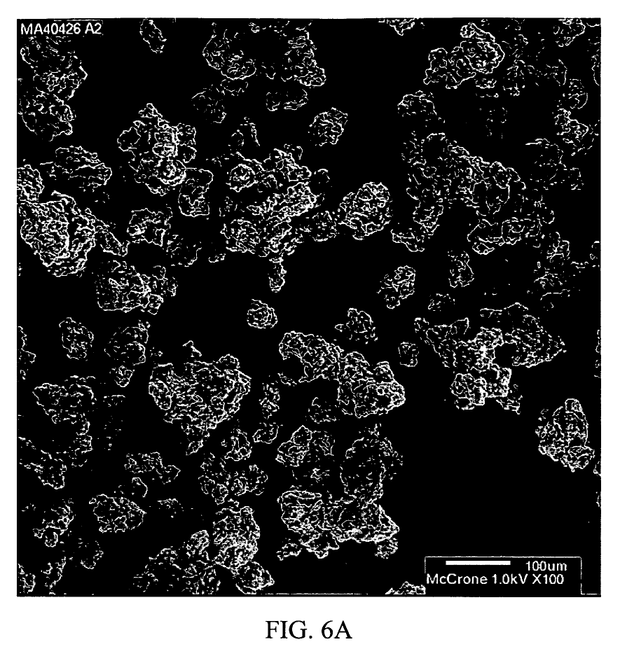

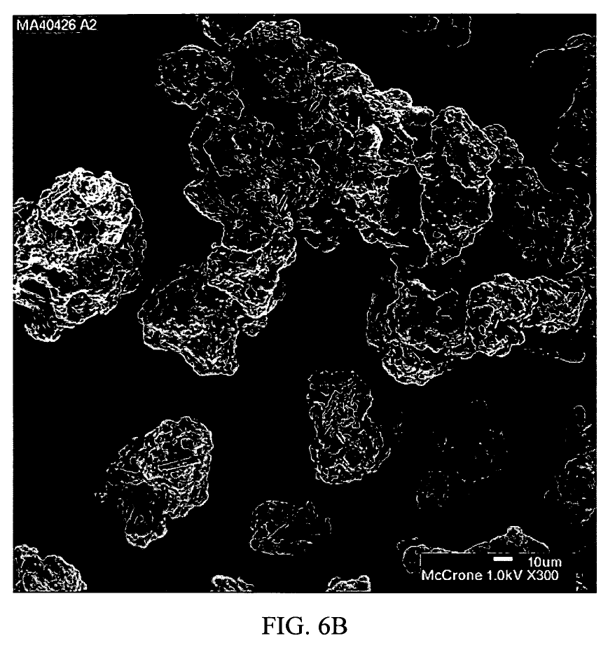

FIG. 6A is an electron micrograph of GeCoated Ramipril API screened to 150 μm through a RoTap #100 mesh at 100-fold magnification.

FIG. 6B is an electron micrograph of GeCoated Ramipril API screened to 150 μm through a RoTap #100 mesh at 300-fold magnification.

FIG. 6C is an electron micrograph of GeCoated Ramipril API screened to 150 μm through a RoTap #100 mesh at 750-fold magnification.

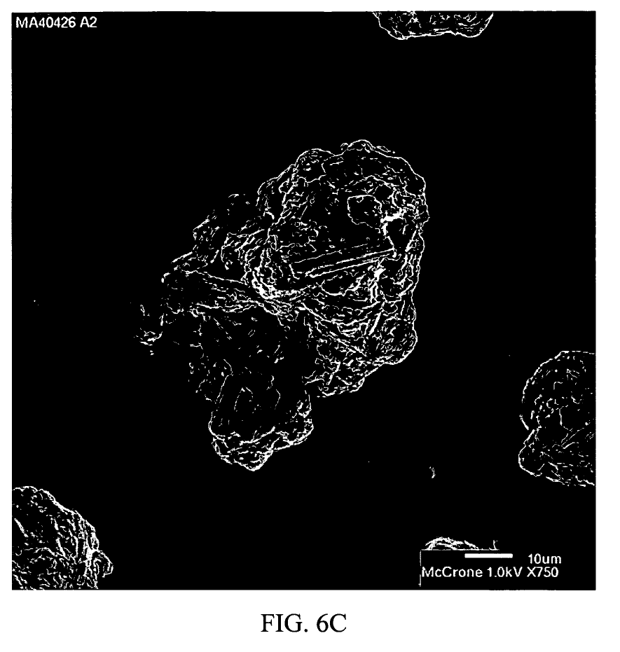

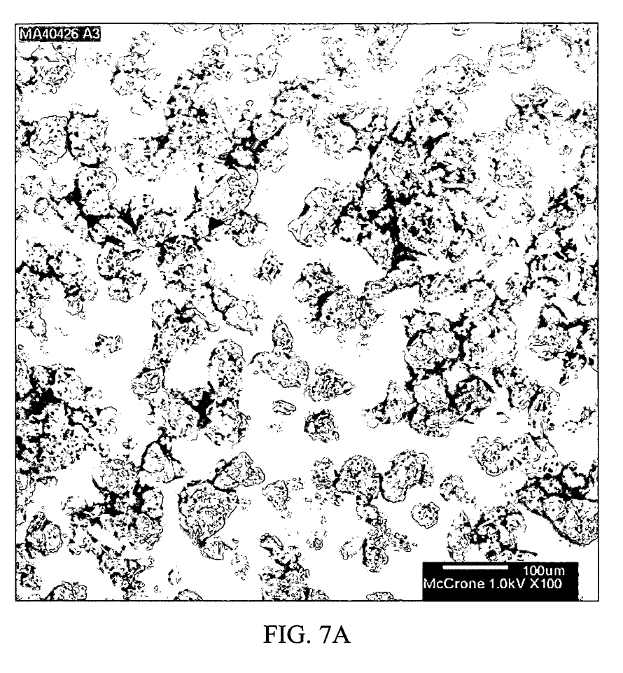

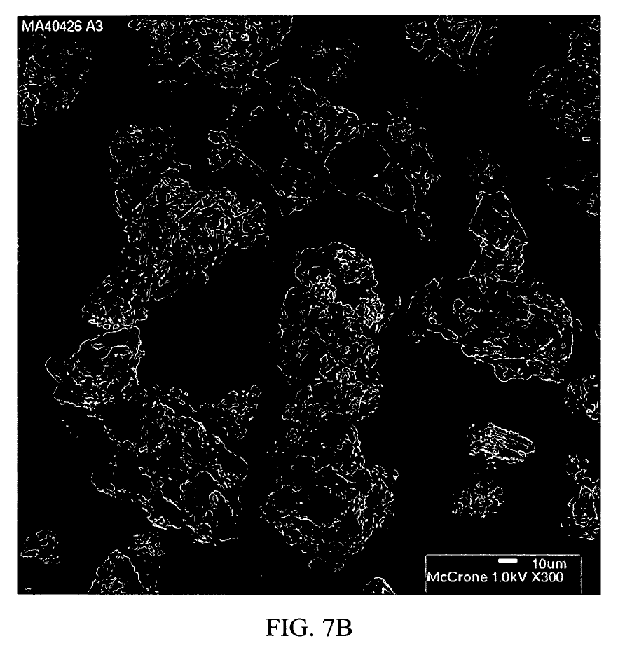

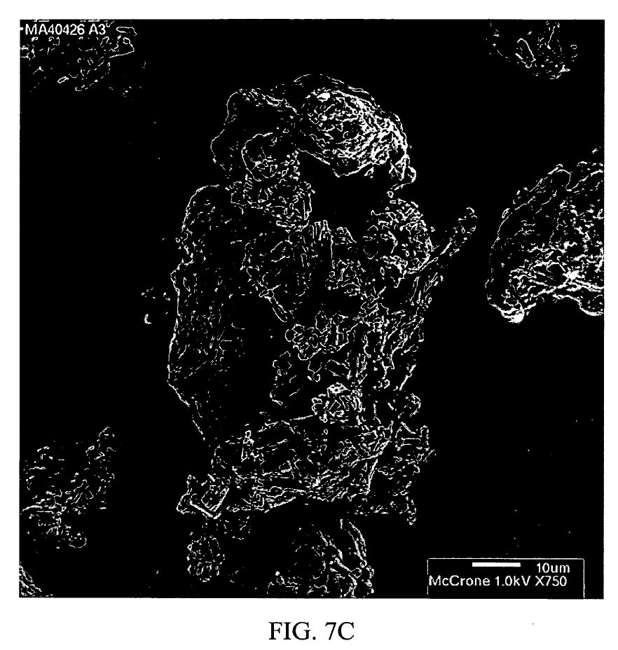

FIG. 7A is an electron micrograph of GeCoated Ramipril API screened to 90 μm through #170 mesh at 100-fold magnification.

FIG. 7B is an electron micrograph of GeCoated Ramipril API screened to 90 μm through #170 mesh at 300-fold magnification.

FIG. 7C is an electron micrograph of GeCoated Ramipril API screened to 90 μm through #170 mesh at 750-fold magnification.

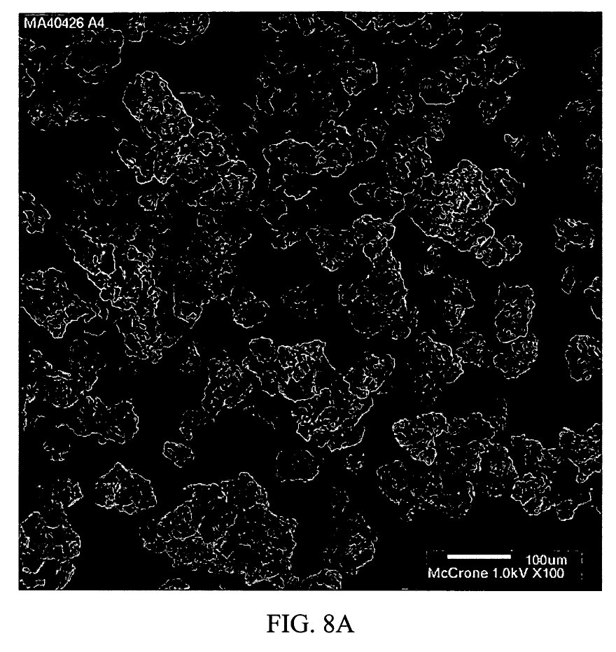

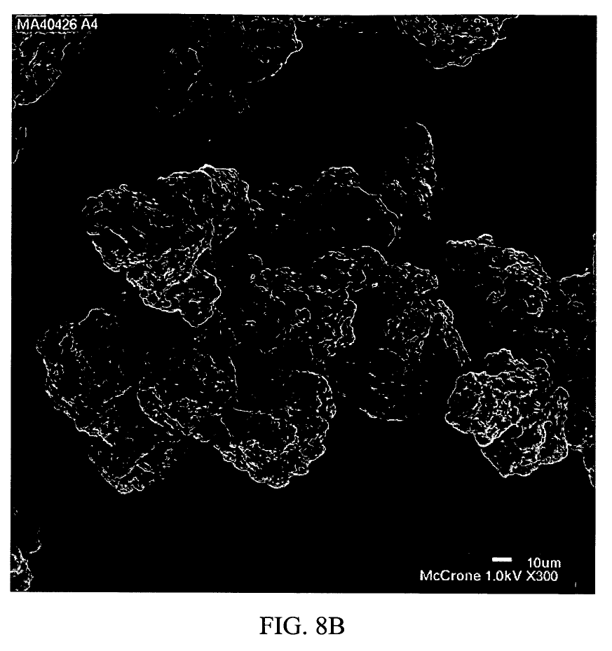

FIG. 8A is an electron micrograph of unscreened GeCoated Ramipril at 100-fold magnification.

FIG. 8B is an electron micrograph of unscreened GeCoated Ramipril at 300-fold magnification.

FIG. 8C is an electron micrograph of unscreened GeCoated Ramipril at 750-fold magnification.

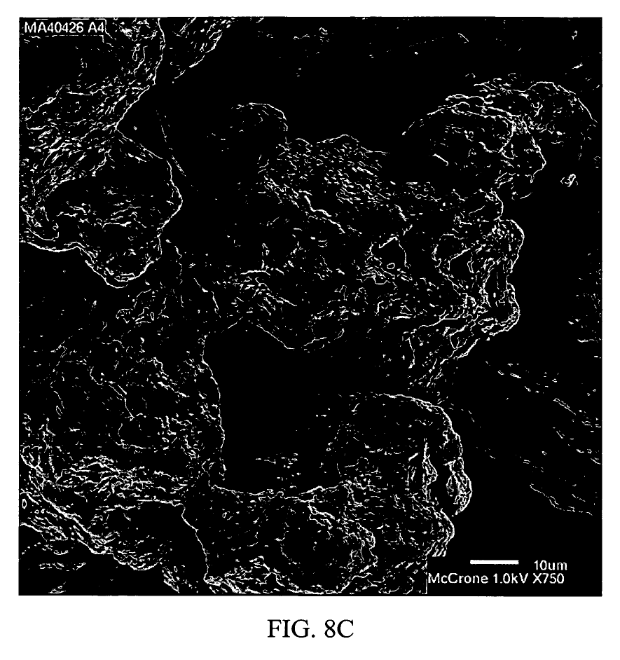

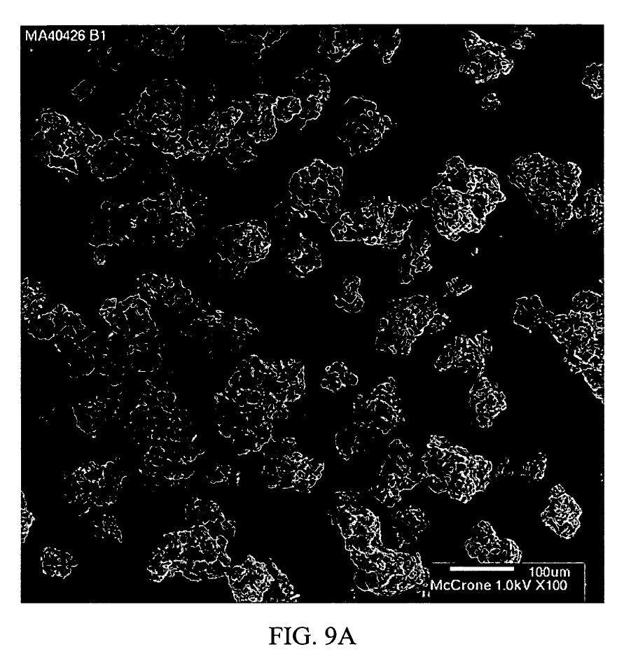

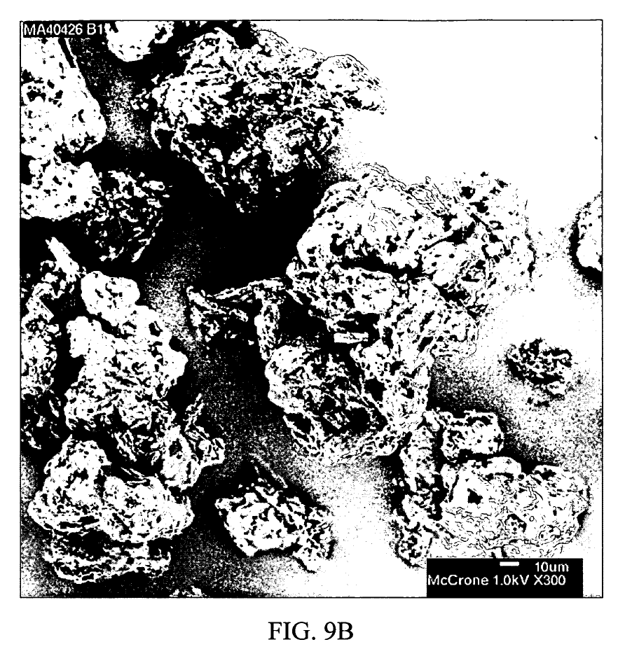

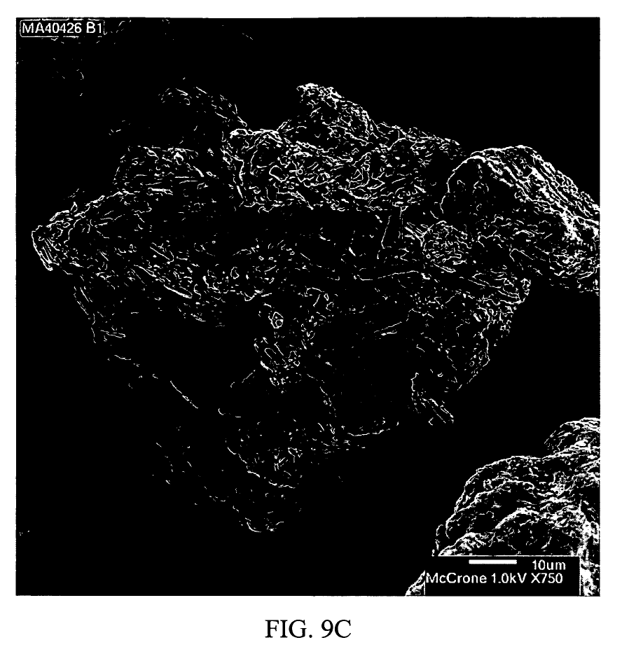

FIG. 9A is an electron micrograph of GeCoated Ramipril at 100-fold magnification screen to 150 μm through #100 mesh.

FIG. 9B is an electron micrograph of GeCoated Ramipril at 300-fold magnification screen to 150 μm through #100 mesh.

FIG. 9C is an electron micrograph of GeCoated Ramipril at 750-fold magnification screen to 150 μm through #100 mesh.

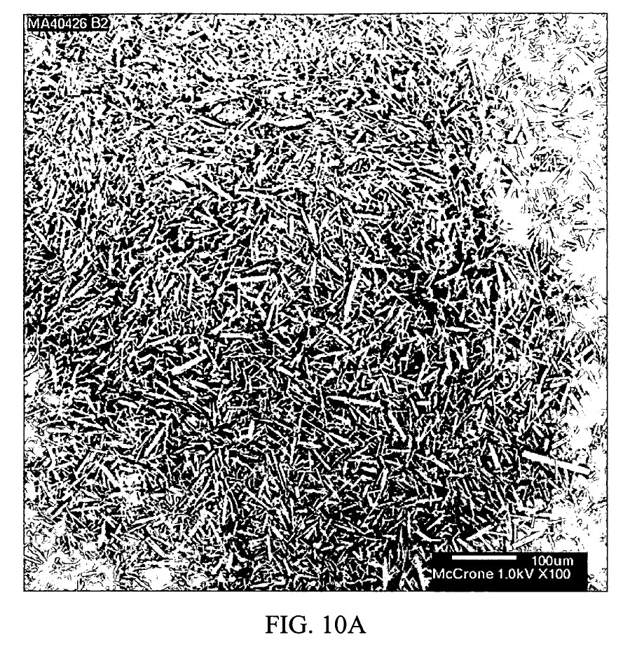

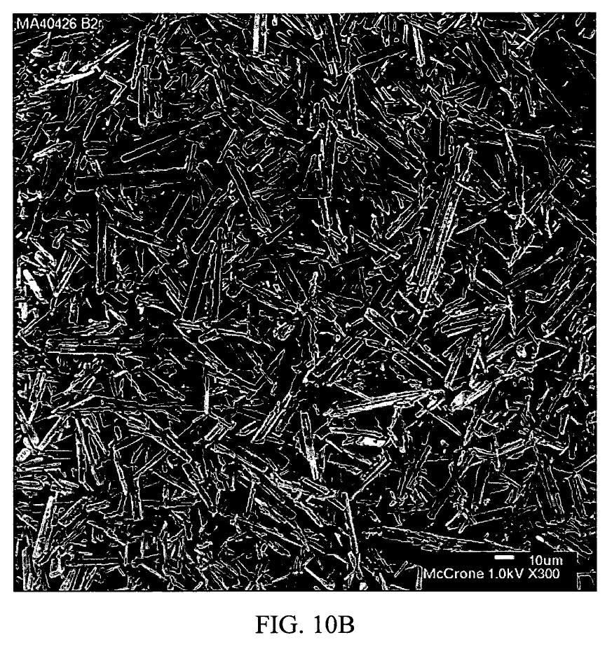

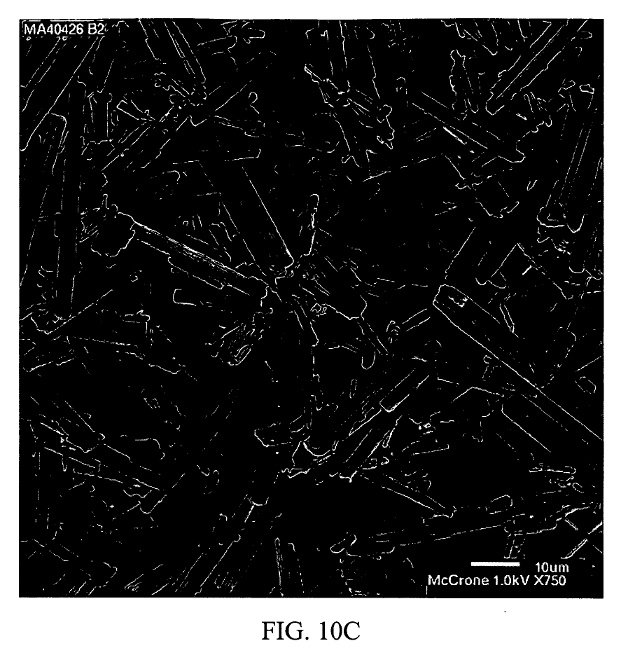

FIG. 10A is an electron micrograph of unscreened ramipril at 100-fold magnification.

FIG. 10B is an electron micrograph of unscreened ramipril at 300-fold magnification.

FIG. 10C is an electron micrograph of unscreened ramipril at 750-fold magnification.

FIG. 11A is a graph that illustrates a linear rate of DKP formation of less than about 0.5% DKP formation after a tested period of 3 months at room temperature and less about 2% DKP formation after an extrapolated period of 36 months at room or ambient temperature from a ramipril tablet produced with individually coated ramipril particles of the present invention.

FIG. 11B is a graph that illustrates a linear rate of DKP formation of less than about 0.5% DKP formation after a tested period of 3 months at room temperature and less about 1.5% DKP formation after an extrapolated period of 36 months at room or ambient temperature from a ramipril tablet produced with individually coated ramipril particles of the present invention.

FIG. 11C is a graph that illustrates a linear rate of DKP formation of less than about 0.5% DKP formation after a tested period of 3 months at room temperature and less about 3% DKP formation after an extrapolated period of 36 months at room or ambient temperature from a ramipril tablet produced with individually coated ramipril particles of the present invention.

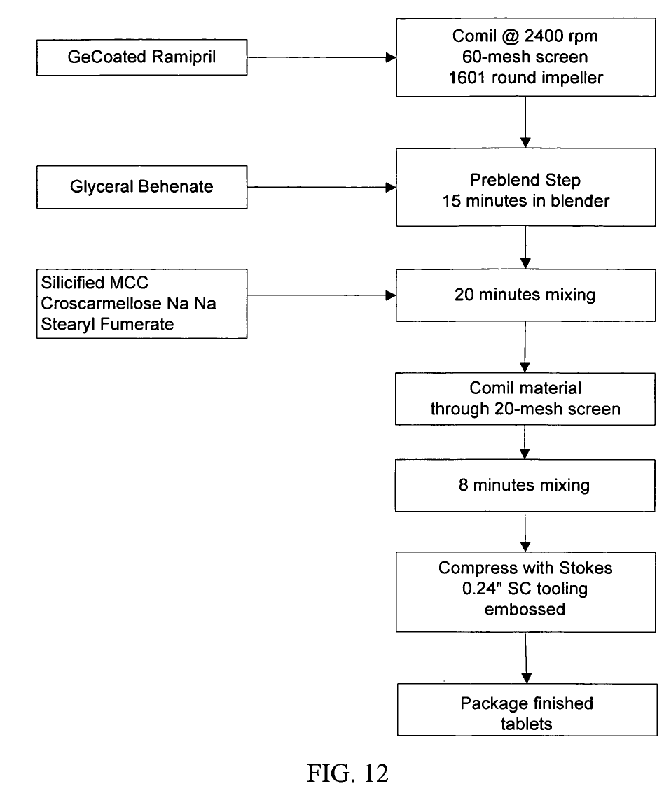

FIG. 12 is a flow chart of a ramipril tablet preparation formulated in accordance with one embodiment of the invention.

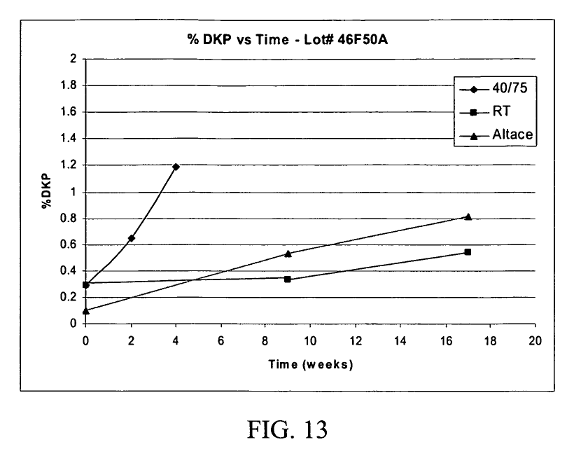

FIG. 13 is a plot of % DKP vs. time for comparative ramipril formulations.

### DETAILED DESCRIPTION

By way of illustration and to provide a more complete appreciation of the present invention and many of the attendant advantages thereof, the following detailed description is given concerning the novel individually coated stabilized ramipril particles, novel anhydrous pharmaceutical grade powders, novel stabilized ramipril pharmaceutical compositions, novel methods for improving ramipril bioavailability, and novel methods of manufacture and stabilization of ramipril formulations.

In general, the present invention employs a pharmaceutical composition that is suitable for oral administration that comprises an effective amount of novel stabilized, individually coated, single ramipril particles contemplated herein to treat or prevent a cardiovascular disorder. While the present invention may be embodied in many different forms, several embodiments are discussed herein with the understanding that the present disclosure is to be considered only as an exemplification of the principles of the invention, and it is not intended to limit the invention to the embodiments described or illustrated.

Definitions

The term “coating”, as used herein, refers to a process for covering or surrounding a single particle with one or more layers of a coat forming material to stabilize the particle. The term “coated”, as used herein, has a somewhat different meaning compared to “coating” and refers to a single or individual particle which is covered with or surrounded by a coat forming material, wherein the coat forming material remains distinct from the single particle that it covers, and with whose aid the particle is stabilized. While the covering by the coat forming material does not necessarily need to be uniform or to cover or surround the entire particle surface, the covering by the coat forming material should be sufficient to impart improved stability to the single particle once coated, as compared to the same uncoated particle. Preferably, but not necessarily, the coat forming material will completely cover or encase the particle in a substantially uniform layer. It is also preferable that the coated particle, when dried, has no substantial gain in moisture relative to its uncoated form.

The term “wet coating”, as used herein, refers to a coating process wherein a particle to be coated is coated in wet form, the process does require dispersing or suspending, but not dissolving, the particle in a continuous liquid phase prior to coating and, at conclusion of the process, the dry coated particle has no substantial gain in moisture relative to its uncoated form.

The term “particle(s)” is used herein generally to refer to a solid, single crystalline particle, irrespective of its size, shape or morphology. Accordingly, the term particle, as used herein, excludes an agglomerate which is a composition that includes single particles gathered together to form a larger particle having varying degrees of open spaces or voids between its individual component particles.

The term “stabilizing”, as used herein, refers to a coating process by which a particle is stabilized.

The terms “stabilized”, “stability” or “stable”, as applied to individually coated, ramipril particles or compositions formulated with same, mean to describe products that are substantially-free of breakdown products or degradants, such as the ramipril-diacid and/or ramipril-DKP, especially under formulation and extended storage conditions. Preferably, the particles remain stable over a period of at least about 36 months from the date that the individual particles are first coated or the compositions are first formulated, and not to the normal metabolic process that occurs when a product, like ramipril, is administered orally and is converted in the body to an active or other form. By way of numerical example, it is believed that, when single ramipril particles are stabilized in accordance with the present invention, the formation of ramipril-DKP over the shelf-life is less than about 0.3% during about the first three months and less than about 3.0% during a period of at least about 36 months from the date that the ramipril particles are first coated. Preferred individually stabilized ramipril particles have a ramipril-DKP formation of less than about 0.3% during about the first three months and less than about 2.0% during such extended period, and more preferred individually stabilized ramipril particles have a ramipril-DKP formation of less than about 0.3% during about the first three months and less than about 1.5% during such extended period. See FIGS. 11A, 11B and 11C. Thus, the individually coated ramipril particles of the present invention provide the basis for novel stabilized ramipril compositions that have remarkably improved stability and biopharmaceutical profiles and are particularly advantageous for oral delivery.

Preferably, the loss of ramipril potency due to ramipril-DKP formation from compositions formulated with the individually coated, single ramipril particles over the shelf-life is less than about 0.04% to about 0.095% on average per month for at least about 36 months from the date that the stabilized ramipril compositions are first formulated. Preferred ramipril solid dosage forms have ramipril-DKP formation of less than about 0.04% to about 0.85% on average per month for such an extended period, more preferred ramipril solid dosage forms have ramipril-DKP formation on the order of less than about 0.04% to about 0.0.055% per month on average for such an extended period, and even more preferred ramipril solid dosage forms have ramipril-DKP formation on the order of less than about 0.04% to about 0.0.042% per month on average for such an extended period.

The terms “diketopiperazine” or “ramipril-DKP” mean diketopiperazine compounds derived from the decomposition or degradation of ramipril. These ramipril-DKP compounds form, as indicated above, as a result of cyclization, condensation and/or breakdown arising from exposure to heat, air, moisture, stress, compaction or other interactions or events.

The term “substantially-free” refers to the stabilized individually coated, ramipril particles and dosage forms described herein that have significantly reduced levels of detectable breakdown products; i.e., ramipril-diacid and/or ramipril-DKP, especially when compared to the levels of detectable breakdown products resulting from the decomposition of ramipril particles in their uncoated state.

The term “cardiovascular disorder(s)”, is used herein broadly and encompasses any disease, illness, sickness, disorder, condition, symptom or issue involving or concerning any part or portion of the heart or blood vessels of an animal, including a human. The term “blood vessel”, as used herein, is defined to include any vessel in which blood circulates. Such cardiovascular disorders include, for example, arterial enlargements, arterial narrowing, peripheral artery disease, atherosclerotic cardiovascular disease, high blood pressure, angina, irregular heart rates, inappropriate rapid heart rate, inappropriate slow heart rate, angina pectoris, heart attack, myocardial infarction, transient ischemic attacks, heart enlargement, heart failure, congested heart failure, heart muscle weakness, inflammation of the heart muscle, overall heart pumping weakness, heart valve leaks, heart valve stenosis (failure-to-open fully), infection of the heart valve leaflets, heart stoppage, asymptomatic left ventricular dysfunction, cerebrovascular incidents, strokes, chronic renal insufficiency, and diabetic or hypertensive nephropathy. These above-listed conditions commonly arise in healthy, pre-disposed or critically ill patients, and may or may not be accompanied by hypertension, angina, light-headedness, dizziness, fatigue or other symptoms.

The terms “treat(s)”, “treated”, “treating” or “treatment” are used herein interchangeably and refer to any treatment of a disorder in an animal diagnosed or inflicted with such disorder and includes, but is not limited to: (a) caring for an animal diagnosed or inflicted with a disorder; (b) curing or healing an animal diagnosed or inflicted with a disorder; (c) causing regression of a disorder in an animal; (d) arresting further development or progression of a disorder in an animal; (e) slowing the course of a disorder in an animal; (f) relieving, improving, decreasing or stopping the conditions of a disorder in a animal; (g) relieving, decreasing or stopping the symptoms caused by or associated with a disorder in an animal; or (h) reducing the frequency, number or severity of episodes caused by or associated with a disorder in an animal.

The terms “prevent(s)”, “prevented”, “preventing” or “prevention” are used herein interchangeably and refer to any prevention or any contribution to the prevention of a disorder in an animal or the development of a disorder if none has occurred in an animal which may be predisposed to such disorder but has not yet been inflicted with or diagnosed as having such disorder.

As indicated above, pharmaceutical compositions according to the present invention will employ a safe and effective amount of stabilized, individually coated, single ramipril particles. The phrase “safe and effective amount(s)”, as used herein, means any amount of a drug which, when administered to a subject to be treated, will achieve a beneficial pharmacological effect or therapeutic improvement consistent with the objectives of the present invention without causing serious, adverse or otherwise treatment-limiting side effects (at a reasonable benefit/risk ratio), within the scope of sound medical judgment. In the case of ramipril, a safe and effective amount may be, for example, an amount that provides some level of inhibition of the ACE enzyme, e.g., in the blood and/or tissue, which is recognized in the art to be therapeutically effective. The beneficial effect will also include at least some decrease in blood pressure for an extended period of time.

Nonetheless, it should be understood that safe and effective amounts of ramipril utilized in accordance with the present invention will vary with the particular cardiovascular disorder, conditions and/or symptoms being treated, the age, weight and physical conditions of the subjects being treated, the severity of the cardiovascular disorder, conditions and/or symptoms, the duration of treatments, the nature of concurrent therapies, the specific dosage form employed, the particular pharmaceutically acceptable carriers utilized, and like factors within the knowledge and expertise of the attending physicians. Exemplary safe and effective amounts of ramipril include those amounts mentioned herein, administered one or more times per day, as will be more fully describe herein below.

It should be understood that the term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range.

As used herein, the terms “comprising,” “comprises”, “comprised of,” “including,” “includes,” “included,” “involving,” “involves,” “involved,” and “such as” are used in their open, non-limiting sense.

It should be understood that the phrase “pharmaceutically acceptable” is used adjectivally herein to mean that the modified noun is appropriate for use in a pharmaceutical product.

The term “pharmaceutically acceptable salt” refers to a salt that retains the biological effectiveness of the free acid and/or base of the specified compound. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Mack Publ. Co., Easton.

The term “derivative” as used herein means a chemically modified compound wherein the chemical modification takes place at one or more functional groups of the compound and/or on an aromatic ring, when present. The derivative may retain the pharmacological activity of the compound from which it is derived.

As to the term “bioavailability”, it is used herein to mean the degree a drug is available to the body. Bioavailability is influenced by how much and the rate at which the drug is absorbed, circulated, distributed, metabolized and excreted.

The term “pharmaceutical grade”, as used herein, means that a substance meets pharmaceutical standards, and that its purity is superior as compared to the purity of the same such substance when classified as food grade, which is less pure.

The term “pharmaceutical grade powder”, as used herein, refers to a powder that is pharmaceutical grade and is least about 98% pure.

As to the term “anhydrous”, it refers to a water content of less than between about 0.9% and 1.1%, and more preferably less than between about 0.7% and about 0.9%, and even more preferably less than about 0.5%.

The term “blending compound” or “blending agent” refers to a waxy substance suitable for co-milling with an ACE inhibitor (e.g., ramipril) which stabilizes the active agent against degradation processes (e.g., ramipril-DKP formation). The ACE inhibitor can be in uncoated form or in agglomerate form (e.g., GeCoated ramipril). Non-limiting examples of blending compounds include glyceryl behenate and other long chain fatty acid-containing glycerol esters.

Ramipril

As discussed and described above, ramipril is an angiotensin converting enzyme (ACE) inhibitor used in the prevention and/or treatment of cardiovascular disorders, especially hypertension and nephropathia, and is one of the most frequently prescribed drugs for congestive heart failure.

Ramipril is an azabicyclo compound. It is known that ramipril is an ester that can form pharmaceutically acceptable salts. References to ramipril, therefore, include the esters and those common salts known to be substantially equivalent to ramipril. Pharmaceutically acceptable salts of ramipril include, for example, salts with pharmaceutically acceptable amines or inorganic or organic acids such as, HCl, HBr, H₂ SO₄, maleic acid, fumaric acid, tartaric acid and citric acid.

It is also known that the molecule corresponding to ramipril has five chiral centers, and that it can occur in 32 different enantiomeric forms. The ramipril ethyl ester is preferred, and the ramipril enantiomer with the chemical name (2S,3aS,6aS)-1 [(S)-N-[(S)-1-carboxy-3-phenylpropyl]alanyl]octahydrocyclo-penta[b]pyrrole-2-carboxylic acid, 1-ethyl ester is most preferred.

Nevertheless, it should be understood by those skilled in this field that ramipril and derivatives thereof may exist in any satisfactory form in accordance with the present invention, e.g., in the form of its racemate or an isomer, namely, a geometric isomer, a structural isomer, an enantiomer, a stereoisomer or a diastereomer, in the non-salt form or in the form of a salt, and in single or multiple forms or in mixtures thereof, and that all such single, multiple, salt and non-salt forms and mixtures thereof are contemplated by the present invention.

The development of new formulations that increase bioavailability and stability of ramipril and derivatives thereof is important in providing safer and more effective drugs to the public. The stabilized, individually coated, single ramipril particles and pharmaceutical grade powders produced therewith are substantially more stable and will allow more effective dosage strengths and combinations of ramipril to be available.

Ramipril Particle Stabilization

The present invention, through single particle coating, produces stabilized, individually coated, ramipril particles that can be used in the manufacture of low dose, dry blend, and direct compression ramipril pharmaceutical products. This invention will allow the small particle size distribution and high surface area (micron sized particles) necessary to achieve content uniformity of low dose ramipril products using dry blend and direct compression technology available today. Examples of a stabilized, individually coated, ramipril particles manufactured in accordance with the present invention are illustrated in FIGS. 1, 2 and 3.

The present invention therefore concerns methods to convert uncoated, ramipril particles into stabilized, individually coated, ramipril particles, which do not agglomerate.

While the present invention contemplates a variety of processes to individually coat the ramipril particles, the invention generally contemplates a process that involves suspending or dispersing ramipril particles in an aqueous liquid phase, into which a coat forming material has been dissolved, to coat the ramipril particles, removing water or drying the aqueous liquid phase to precipitate the individually coated, ramipril particles from the aqueous liquid phase, and collecting the precipitated individually coated, ramipril particles to form the novel, anhydrous pharmaceutical grade ramipril powders. Nevertheless, the coat forming material may be applied by any suitable coating technique, so long as the individually coated, ramipril particles do not agglomerate prior to being individually coated.

Examples of such wet coating processes or techniques contemplated by the present invention include spray-drying, turbo drying, spray congealing, pan coating, disk spinning, fluidized bed coating, crystallization, cryogenation, super critical fluid extraction, nanoencapsulation, and coacervation. Spray-drying methods, however, are preferred.

When spray-drying is the selected method to individually coat the ramipril particles in accordance with the present invention, the discrete crystalline ramipril particles are spray-dried with a spray apparatus that uses a feed solvent which is a suspending medium in which the discrete ramipril particles are practically insoluble.

A typical spray-drying apparatus for use in accordance with the present invention comprises a drying chamber, atomizing means for atomizing a feed solvent introduced into the drying chamber, a source of heated drying gas that flows into the drying chamber to remove solvent from the atomized-feed solvent and product collection means located downstream of the drying chamber. Examples of such spray dryers include Buchi Model B290, Brinkmann Instruments, Westbury, N.Y., and Niro Models PSD-1, PSD-2 and PSD-4, Niro A/S, Soeborg, Denmark.

In the following discussion, it is assumed that the spray-drying apparatus is cylindrical. However, the dryer may take any other shape suitable for spray-drying a feed solvent, including square, rectangular, and octagonal. The spray-drying apparatus is also depicted as having one atomizing means. However, multiple atomizing means can be included in the spray-drying apparatus to achieve higher throughput of the feed solvent.

An exemplary drying apparatus comprises a drying chamber, a drying chamber top, a collection cone, a connecting duct connected to the distal end of the collection cone, a cyclone and a collection vessel. An atomizer is shown has a feed solvent. Drying gas from a drying gas source is introduced through drying gas inlets, typically via an annular opening in drying chamber top, in a flow direction that is not parallel to the atomized droplet flow which is typically introduced vertically at the center of the top of the dryer via atomizing means. The non-parallel drying gas flow typically has an inward vector that is toward the atomized droplets near the center of the chamber and a radial vector that is an off-center flow. Drying gas introduced in this manner induces flow that is circular (generally parallel to the circumference of the cylindrical chamber), and that creates circulation cells that carry droplets or particles initially downward and then back up to the drying chamber top so as to cause a large fraction to pass near drying gas inlet and atomizing means. Such flow introduces rapid and turbulent mixing of the drying gas and atomized feed-solvent, leading to rapid drying of the droplets to form the stabilized, individually coated, single ramipril particles. The individually coated, single ramipril particles are entrained by the drying gas through collection cone to connecting duct, and then to cyclone. In the cyclone, the individually coated, single ramipril particles are separated from the drying gas and evaporated solvent, allowing the particles to be collected in collection vessel. Instead of a cyclone, a filter may be used to separate and collect the stabilized, individually coated, single ramipril particles from the drying gas and evaporated solvent.

The drying gas may be virtually any inert gas, but to minimize the risk of fire or explosions due to ignition of flammable vapors, and to minimize undesirable oxidation or other adverse interactions with ramipril, the coat forming material or other materials in the dispersion or suspending medium, an inert gas such as air, nitrogen, nitrogen-enriched air, or argon is utilized. The temperature of the drying gas at the gas inlet of apparatus for aqueous suspending medium is typically from about 90° C. to about 140° C., and preferably is between about 100° C. to about 125° C. The temperature of the product particles, drying gas, and evaporated solvent at the outlet or distal end of collection cone typically ranges from about 0° C. to about 100° C., and preferably is between about 50° C. and 60° C. for same aqueous medium.

In accordance with the present invention, the ramipril particles, wherein each particle is individually coated with a coat forming material, are formed with rapid solidification of the atomized droplets. To accomplish this, an apparatus is equipped with atomizing means such as, but not limited to a two-fluid nozzle, a single fluid nozzle, rotating disk nozzle, ultrasonic nozzle or similar, that produces relatively small droplets, generally with median diameters between about 5 μm to 1000 μm, and typical average droplet diameters of between about 5 μm to about 300 μm. In a two-fluid nozzle, the feed solvent is mixed with an atomizing gas, such as air or nitrogen, atomizing the feed into small droplets. This small droplet size, along with the turbulent mixing of a portion of the drying gas within the nozzle as well as at the outlet of the nozzle, results in a large surface area and driving force for evaporation of the solvent from the droplet, leading to rapid removal of solvent from the droplet. The resulting stabilized, individually coated, single ramipril particles may have a median particle size similar to the original starting material, and additionally of about 99% no more than 300 μm.

When a pressure nozzle is used in a conventional spray-dryer apparatus, the resulting non-parallel flow creates circulation cells as described above that causes rapid and turbulent mixing of the drying gas and atomized spray solution, leading to rapid drying of the larger droplets. This approach has the benefit of allowing the larger droplets formed by pressure nozzles to be dried in a conventional-sized drying chamber. As a result, homogeneous solid, stabilized, individually coated, ramipril particles may be successfully made in this manner.

In the drying chamber, production of solid, stabilized, individually coated, ramipril particles, can be accomplished by properly disposing the pressure nozzle within the drying chamber, considering the height, width and overall design of the drying chamber. Preferably, the height and width of the drying chamber should allow sufficient minimum distance for a droplet to travel before impinging on a surface of the drying chamber. Such adjustments and considerations are within the purview of those of skill in the art.

While the height and width of the drying chamber is important to determining the minimum distance a droplet travels before impinging on a surface of the drying apparatus, it should be understood that the volume of the drying apparatus is also important. The capacity of a spray-dryer is determined, in part, by matching the flow rate of the feed solvent to the temperature and flow of the drying gas. Simply stated, the temperature and flow rate of the drying gas must be sufficient so that sufficient temperature for evaporating the feed solvent is delivered to the spray-drying apparatus. Thus, as the flow rate of the feed solvent is increased, the flow rate and/or temperature of the drying gas may be increased to provide sufficient energy for formation of the desired product. Since the allowable temperature of the drying gas is limited by the chemical stability of ramipril dispersed or suspended in the feed solvent, the drying gas flow rate should be adjusted to allow for an increased capacity, i.e., increased flow of the feed solvent, of the spray-drying apparatus. For a spray-drying apparatus with a given volume, an increase in the drying gas flow rate may result in a decrease in the average residence time of droplets or particles in the dryer, which could lead to insufficient time for evaporation of solvent from the droplets to form the solid, stabilized, individually coated, ramipril particles prior to impinging on a surface in the dryer. As a result, the volume of the spray dryer should be sufficiently large that the droplet is sufficiently dry by the time it impinges on any of the internal surfaces of the dryer to prevent build-up of material. This requires a balance of the atomization gas pressure, the size of the orifice in the spray nozzle for the feed solvent and the atomization gas, the feed solvent flow rate, and the temperature and flow rate of the drying gas. The temperature of the atomization gas may also be altered to achieve the specific desired results. As will be apparent to those of skill in the art the average residence time should be sufficient to ensure that the droplets are dry prior to impinging on a surface of the spray drier.

This spray-drying process by which solid, stabilized, individually coated, ramipril particles are produced is discussed further in the examples below.

According to one embodiment, the individual ramipril particles are prepared in the form of stabilized, individually coated, single crystalline particles. The single ramipril crystalline particles are each coated individually with a coat forming material, such as hydroxypropyl methyl cellulose (HPMC), polyvinylpropropylene, starch, stearate, silica or the like, without agglomerate formation prior to individual coating, as further discussed below.

Preferably, the applied coatings, once dried, have a thickness to effectively stabilize the individually coated, single ramipril particles. The individually coated, single ramipril particles of the present invention may have a bulk density of about 0.22 gm/ml, a tapped density of about 0.27 gm/ml, a Carr's Index equal to about 18.5% and a mean particle size of about 74.7 μm.

Preferably, the particle size distribution of the individually coated, single ramipril particles is representative of the original starting material, and additionally may be between about 876 μm to about 3.9 μm; preferably, a particle size distribution wherein at least about 75.0% of the individually coated, single ramipril particles have a size distribution of less than about 50 μm; and even more preferably, a particle size distribution wherein at least about 50.0% of the individually coated, single ramipril particles will have a particle size of less than 20 μm. Alternatively, a particle size distribution of individually coated, single ramipril particles (e.g., spray-dried particles—wet coating), as contemplated by the present invention, may be as follows: (a) about 80.0%—less than about 20 μm; (b) about 15%—between about 20 μm and about 50 μm; (b) about 1.5% between about 50 μm and 150 μm; and (d) about 1.0%—between about 150 μm and 538 μm.

The coat forming material is preferably a polymer coating, such as a HPMC, e.g., Methocel E5 Prem LV, in the form of a liquid coating, that is sprayed onto the ramipril particles or in which the ramipril particles are suspended and then spray-dried via, for example a spray dryer. Generally speaking, to form this polymer liquid coating the HPMC is first dissolved in about 5, 10 or 15% or more of the amount of ramipril in water to obtain a final dispersion, such as about 30% solids wt/wt, 20% solids wt/wt, 10% solids wt/wt; using about 30% as a starting point to determine and obtain the desired viscosity suitable for pumping and atomization. The prepared coating liquids are preferably, but not necessarily, water-based dispersions due to environmental concerns. Thus, organic based dispersions are also contemplated by the present invention, so long as the single ramipril particles remain suspended or dispersed, not dissolved, therein.

The spray-dried product formed by the methods of the present invention comprises single ramipril particles individually coated with a coat forming material. It should be understood that all ramipril particles, before and after coating, are in a single, solid crystalline state. The amounts and structure of the coated ramipril particles may be measured or viewed by Powder X-Ray Diffraction (PXRD), Scanning Electron Microscope (SEM) analysis, as shown in FIGS. 1, 2 and 3, differential scanning calorimetry (DSC), or any other standard quantitative measurement. Not withstanding the typical agglomeration, clumping, and sticking particles typically undergo; these particles may be separated and still maintain their protective coating unlike particles that are granulated together with said polymers or similar protective substances.

The solid, individually coated, ramipril particles formed may contain from about 50 wt % to about 99 wt % ramipril, or from about 75 wt % to about 95% wt %, or from about 85 wt % to about 95 wt %, depending on the effectiveness of and coating thickness produced by the coat forming material selected.

Coat Forming Material

While the present invention contemplates any suitable material for individually coating the ramipril particles to improve stability and bioavailability, the coat forming material should be inert, in the sense that it does not chemically react with the ramipril particles in an adverse manner, and it should not cause the ramipril particles to agglomerate prior to their being individually coated. The coat forming material can be neutral or ionizable; however, it is critical to the invention that the coat forming material does not solubilize the ramipril particles when mixed together to form the feed solvent prior to coating by, for example, spray-drying.

The material is a “coat forming material” in accordance with the present invention if it meets at least the one of the following conditions, preferably at least four of the following conditions, and most preferably all eight of the following conditions. The first condition is that the coat forming material improves the stability of the single ramipril particles against decomposition into ramipril-DKP and ramipril-diacid degradants under formulation and storage conditions to such an extent that the individually coated, ramipril particles are substantially-free of such degradants, as compared to single ramipril particles formulated and stored under identical conditions, but in their uncoated state. Preferably, the coat forming material improves the ramipril stability to an extent that the formation of additional ramipril-DKPs in pharmaceutical compositions employing such stable ramipril particles over the shelf-life of such compositions is less than about 0.3% during about the first three months and less than a total of about 4.0% during a period of at least about 36 months from the date that such compositions are first formulated, or more preferably to less than a total of about 3.0% during a period of at least about 36 months from the date that such compositions are first formulated, or more preferably to less than a total of about 2.0% during a period of at least about 36 months from the date that such compositions are first formulated, or more preferably to less than a total of about 1.5% during a period of at least about 36 months from the date that such compositions are first formulated.

The second condition is that the coat forming material does not dissolve or interact adversely with the ramipril particles during or after the spray-drying or other coating processes.

The third condition is that the coat forming material sufficiently coats each ramipril particle individually to stabilize the single ramipril particle following the coating process under formulation and shelf-life conditions.

The fourth condition is that the coat forming material and coating process selected does not cause the individual ramipril particles to agglomerate before each ramipril particle is adequately coated. In other words, following the coating and drying process, the individually coated, single ramipril particles preferably remain as individual, discrete particles, but in a coated state.

The fifth condition is that the coat forming material when applied to a particle is in intimate contact with the particle or another layer in contact with the particle.

The sixth condition is that the coat forming material will encase the particle under conditions when the particle is in solid form at temperatures below the melting or degradation temperature of the coat forming material, and wherein the coat forming material remains distinct from the particle that it encases.

The seventh condition is that the coat forming material will uniformly encase each ramipril particle.

The eighth condition is that the coat forming material does not substantially alter the particle size distribution of the individually coated, single ramipril particles as compared to the particle size distribution of the uncoated single ramipril particles used as the starting materials. In other words, the particle size distribution of the solid, individually coated, single ramipril particles should mimic or resemble the particle size distribution of the uncoated single ramipril particles.

Examples of coat forming materials contemplated by the present invention include polymers, starches, stearates, silicas, waxes (atomized glyceryl palmitostearate, dioctyl sodium sulphosuccinate), surfactants, and fatty acids (preferably having a chain length of eight carbons or greater which may contain one or more double bonds).

Starches that may be suitable for use as coat forming materials in the present invention include pregelatinized starch, namely, PCS® PC-10, Asahei Kasei, a modified corn starch, e.g., Pure-Cote™ B793, Grain Processing Corp. and an unmodified high amylase corn starch, such as Hylon® VII, National Starch and Chemicals.

A stearate that may be suitable for use as a coat forming material is atomized glyceryl palmitostearate, Precirol® ato 5, Gattefosse s.a., France.

Polymers that may be suitable for use with the present invention include cellulosic or non-cellulosic polymers. The polymers may be neutral or ionizable in aqueous solution. Of these, ionizable and cellulosic polymers are preferred, with cellulosic polymers being more preferred.

The term “polymer” is used herein in the generic sense and refers to molecules that are formed with a linked series of repeating simple or different monomers, and may include, for example, single polymers, co-polymers, block polymers including tri-block polymers and block co-polymers, self assembling polymers such as macromonomers that form nanotubes, hydrophilic and hydrophobic polymers, and the like. Polymers in accordance with the present invention may be selected from a broad range of polymer-forming materials, such as polysaccharides, celluloses, and organic moieties such as polyvinyl pyrrolidines and plastics.

Examples of cellulose derivatives suitable for protective coatings include hydroxypropylcellulose, hydroxypropylmethylcellulose, hydroxylpropyl-methylcellulose, hydroxyethylcellulose, ethylcellulose, cellulose acetate phthalate, cellulose acetate, polyvinyl acetate phthalate, polyvinylpyrrolidone, cationic and anionic polymers, copolymers with neutral character based on poly(meth)acrylic esters (Eudragit® E, Eudragit® E 30 D), anionic polymers of methacrylic acid and methyl methacrylate (Eudragit®L or S, Eudragit®L 30 D), and gelatin.

Examples of cellulose based ionizable polymers include hydroxypropyl-methyl cellulose acetate succinate, hydroxypropyl methyl cellulose succinate, hydroxylpropyl cellulose acetate succinate, hydroxyethylmethyl cellulose succinate, hydroxyethyl cellulose acetate succinate, hydroxypropylmethyl cellulose phthalate, hydroxethylmethyl cellulose acetate succinate, hydroxyethylmethyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methylcellulose acetate succinate phthalate, hydroxypropylmethyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropylmethyl cellulose acetate trimellitate, hydroxypropyl cellulose acetate trimelllitate succinate, cellulose propionate trimellitate, cellulose butryrate trimellitate, cellulose acetate terephthalate, cellulose acetate isophthalate, cellulose acetate pyridine dicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid, cellulose acetate and ethyl picolinic acid cellulose acetate.

Additional polymers include non-ionizable cellulosic polymers comprising hydroxypropyl methyl cellulose acetate, hydroxypropyl methylcellulose, hydroxypropyl cellulose, methyl cellulose, hydroxyethyl methyl cellulose, hydroxyethyl cellulose acetate, and hydroxyethyl ethyl cellulose.

Another class of polymers that may be suitable for use with the present invention concern non-cellulosic polymers that are amphiphilic is copolymers of a relatively hydrophilic and a relatively hydrophobic monomer. Examples include acrylate and methacrylate copolymers. Exemplary commercial grades of such copolymers include the EUDRAGIT® series, which are copolymers of methacrylates and acrylates.

Another class of polymers that may be suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates, such as the Eudragit® series manufactured by Rohm Tech Inc., Malden, Mass., amine-functionalized polyacrylates and polymethacrylates, proteins such as gelatin and albumin, and carboxylic acid functionalized starches such as starch glycolate.

Another class of polymers that may be suitable for use with the present invention comprises non-ionizable (neutral) non-cellulosic polymers, including carboxylic acid functionalized polymethyacrylates, carboxylic acid functionalized polyacrylate, amine-functionalized polyacrylates, amine-functionalized polymethacrylates, proteins, and carboxylic acid functionalized starches. Exemplary polymers include: vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, and polyoxyethylene-polyoxypropylene copolymers.

The polymers may also have hydroxyl-containing repeat units, alkylacyloxy-containing repeat units, or cyclicamido-containing repeat units; polyvinyl alcohols that have at least a portion of their repeat units in the unhydrolyzed form; polyvinyl alcohol polyvinyl acetate copolymers; polyethylene glycol, polyethylene glycol polypropylene glycol copolymers, polyvinyl pyrrolidone polyethylene polyvinyl alcohol copolymers, and polyoxyethylene-polyoxypropylene block copolymers. Within these vinyl copolymers, the second polymer may contain (1) hydroxyl-containing repeat units; and (2) hydrophobic repeat units.

Examples of lipophilic polymers include hydroxy methyl cellulose, hydroxy ethyl cellulose, hydroxy propyl cellulose, hydroxy butyl cellulose, and hydroxyalkyl celluloses such as hydroxy ethyl methyl cellulose, hydroxypropyl cellulose, carboxylmethyl cellulose, carboxyethyl cellulose and corresponding salt and esters.

Inter-polymer complexes may be formed from linear or cross-linked hydrophilic polymers and, in general, are formed from alginate alkyl, alkyl, and hydroxyalkyl celluloses, carrageenan, a variety of types of cellulose, gums, methyl vinyl ether/maleic and hybrid co-polymers, pectins, polyacrylamides, polyethylene glycol, polyvinyl alcohol, polyvinyl acetate, polyvinyl pyrrolidone, starches, styrene/maleic hydride, and similar materials.

Natural, as well as synthetic and semi-synthetic polymeric coatings may be used and include such substances as alginic acid, its alkali metal and ammonium salt, carageenans, galactosamine, gum tragacanth (arabic), guar gum, gummi arabicum, guar gummi, xanthan gummi, pectins, i.e., sodium carboxymethylamino pectin, chitosan, polyfructans, inulin, polyacrylic acids, polymethacrylic acids, methacrylate copolymers, polyvinyl alcohol, polyvinyl pyrrolidene, copolymers of polyvinyl pyrrolidone with vinyl acetate, polyalkylene, and copolymers such as ethylene oxide with propylene oxide. Solid carriers may act as encapsulation coats.

While specific polymers have been discussed as being possibly suitable for use in the feed solvents formable by the present invention, blends of such polymers may also be suitable. Thus, the term “coat forming material”, as used herein, is intended to include blends of polymers or other coat forming materials in addition to a single species of polymer.

Additionally, coat enhancing materials such as, but not limited to plastersizers can be added to the coat forming material. Suitable coat enhancers include, but are not limited to, triethyl citrate (TEC). Preferably the coat enhancing materials does not contribute or facilitate ramipril to degrade in to ramipril-DKP and ramipril diacid.

The amount of coat forming material relative to the amount of ramipril present in the spray-dried particles formed by the present invention depends on the coat forming material and may vary widely from a ramipril-to-polymer weight ratio of from about 99:1 to about 1:1. However, in most cases, except when the drug dose is quite low, e.g., 25 mg or less, it is preferred that the ramipril-to-polymer ratio is greater than about 2:1 and less than about 99:1.

Preferably the coat forming material completely coats the individual ramipril particles; however, so long as the coat forming material coats enough of the surface of the individual ramipril particles to prohibit or slow the degradation of the individual ramipril particles during the process and storage of ramipril compositions then the ramipril is sufficiently coated. In accordance with the present invention the coat forming material can coat between about 85% to 100% of the surface of the individual ramipril crystals. Preferably, the coat forming material coats between about 90% to 100% or between about 95% to 100% or between about 98% to 100%.

The coat forming material can form a coating around the individual ramipril crystals of any thickness so long as the ramipril is substantially-free from degradant products and the desired bioavailability of ramipril is achieved. The coat forming material can form a coating that is between about 0 μm to 1000 μm thick. The coating thickness can be between about 50 μm to 900 μm or between about 100 μm to 800 μm. Preferably, the coating thickness is between about 200 μm to 700 μm.

In general, regardless of the ramipril dose, enhancements in ramipril stability and relative bioavailability increase with decreasing ramipril-to-polymer weight ratio. However, due to the practical limits of keeping the total mass of a solid oral dosage form, e.g., tablet, caplet, capsule or tablet-filled capsule low, it is often desirable to use a relatively high ramipril-to-polymer ratio as long as satisfactory results are obtained. The maximum and minimum ramipril to polymer ratios that yield satisfactory results will vary from polymer to polymer and is best determined in vitro and/or in vivo dissolution or other satisfactory tests known to those versed in this art.

In general, and dependent upon the coat forming material selected, to maximize ramipril stability and/or relative bioavailability, lower ramipril-to-polymer ratios may be needed. At low ramipril-to-polymer ratios, there should be sufficient coat forming material available in the feed solvent to ensure adequate uniform coating of the individual ramipril particles from the feed solvent and, thus, ramipril stability and bioavailability may be much higher. For high ramipril-to-polymer ratios, not enough coat forming material may be present in the feed solvent and inadequate coating may occur more readily. However, the amount of coat forming material that can be used in a solid oral dosage form derived from the individually coated ramipril particles in accordance with the present invention may be limited by the maximum total mass of a solid oral dosage form that is acceptable. For example, when oral dosing to a human is desired, at low ramipril-to-polymer ratios the total mass of ramipril and polymer may be unacceptably large for delivery of the desired dose in a single tablet or capsule. Thus, it may be necessary to use ramipril-to-polymer ratios that are less than those which yield maximum ramipril stability and/or bioavailability in specific dosage forms to provide a sufficient ramipril dose in a solid oral dosage form that is small enough to be easily delivered to a use environment. Of course, it should be understood that it is preferred to utilize a coat forming material, such as Methocel E5 Prem LV, that can accomplish both, i.e., maximum ramipril stability and/or bioavailability in specific dosage forms, under formulation and storage conditions, at a ramipril-to-polymer ratio that provides an effective ramipril dose in a solid oral dosage form that is small enough to be easily delivered to a use environment.

Administration

A preferred form for administration is a solid oral dosage form, such as capsules, tablets, pills, granules, puvules and the like. Other forms of the drug may be in suppositories, suspensions, liquids, powders, creams, transdermal patches, and depots. The drug is conventionally admixed with a pharmaceutically acceptable excipient or inert carrier, such as sucrose, starch, lactose or combinations of various fillers, as discussed below. Of course, other ingredients may also be added, including flavorings, inert diluents, or binders as further discussed below.

The dosage of active ingredient in the compositions of the invention may be varied however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets than being followed by a patient, concurrent medication, and other factors, recognized by those skilled in the art. Based upon the foregoing, precise dosages depend on the condition of the patient and are determined by discretion of a skilled clinician. Generally, ramipril daily dosage levels of between about 0.010 to about 1.5 mg/kg of body weight are administered daily to mammalian patients, e.g., humans having a body weight of about 70 kg. The ramipril dosage range will generally be about 1.25 mg to 50 mg per patient per day, administered in single or multiple doses. Preferably, the dosage range will be between about 1.25 mg to about 25 mg per patient per day; more preferably about 2.5 mg to about 25 mg per patient per day, and most preferably about 5 mg to about 20 mg per day.

Compositions

In formulating the compositions of the present invention, the individually coated, stand alone, ramipril particles, in the amounts described herein, are compounded according to accepted pharmaceutical practice with any pharmaceutically acceptable additives into any suitable type of unit dosage form. Suitable additives include diluents, binders, vehicles, carriers, excipients, disintegrating agents, lubricants, swelling agents, solubilizing agents, wicking agents, cooling agents, preservatives, stabilizers, sweeteners, flavors, etc. While any pharmaceutically acceptable additive is contemplated by the present invention, it should be understood that the additive(s) selected for compounded with the individually coated, stand alone, ramipril particles should not defeat the stability objectives of the present invention.

Examples of excipients include acacia, alginic acid, croscarmellose, gelatin, gelatin hydrosylate, mannitol, plasdone, sodium starch glycolate, sorbitol, sucrose, and xylitol. For molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of additional stabilizers or preservatives include, for example, parahydroxybenzoic acid alkyl esters, antioxidants, antifungal agents, and other stabilizers/preservatives known in the art.

Examples of coloring agents include, for example, water soluble dye, Lake dye, iron oxide, natural colors, titanium oxide, and the like.

Examples of diluents or fillers include water-soluble and/or water-insoluble tabletting fillers. The water-soluble diluent agent may be constituted from a polyol of less than 13 carbon atoms, in the form of directly compressible material (the mean particle size being between about 100 and about 500 microns), in the form of a powder (the mean particle size being less than about 100 microns) or a mixture thereof. The polyol is preferably chosen from the group comprising of mannitol, xylitol, sorbitol and maltitol. The water-insoluble diluent agent may be a cellulosic derivative preferably microcrystalline cellulose. Especially preferred diluents are those with minimal moisture content, such as lactose monohydrate and magnesium oxide.

Examples of disintegrating agents include, but are not limited to, crosslinked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearylfumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A portion of the lubricant may be used as an internal solid lubricant which is blended and granulated with other components of the granulation. Another portion of the lubricant may be added into the final blended material just before compression or encapsulation that coats the outside of the granules in the final blend.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Additional illustrations of adjuvants which may be incorporated in the tablets are the following: a binder such as gum tragacanth (arabic), acacia, corn starch, potato starch, alginic acid, povidone, acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, lactose monohydrate, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminium silicate, maltodextrin, methylcellulose, hydroxypropylcellulose, polyethylene, polyethylene oxide, polymethacrylates, plasdone, sodium alginate, starch, pregelatinized starch, zein or the like; a sweetening agent such as sucrose, potassium acesulfame, aspartame, lactose, dihydrochalcone neohesperidine, saccharin, sucralose, polyols such as xylitol, mannitol, and maltitol, sodium saccharide, Asulfame-K, Neotame®, glycyrrhizin, malt syrup and combinations thereof; a flavoring such as berry, orange, peppermint, oil of wintergreen, cherry, citric acid, tartaric acid, menthol, lemon oil, citrus flavor, common salt, and other flavors known in the art.

The flavoring is advantageously chosen to give a combination of fast onset and long-lasting sweet taste and get a “round feeling” in the mouth with different textures or additives. Cooling agents can also be added in order to improve the mouth feeling and provide a synergy with flavors and sweetness. Various other materials may be present as coatings or to otherwise modify the physical form of the dosage unit. For instance, tablets or capsules may be coated with shellac, sugar or both.

Examples of qualitative stabilized ramipril pharmaceutical compositions contemplated by the present invention comprises solid, individually coated, single ramipril particles, as described herein, admixed with, for example, Ceolus®, lactose, anhydrous lactose DT, lactose monohydrate, starch, spray-dried mannitol (Pearlitol 200 SD), Prosolv® SMCC 50, Prosolv® SMCC 90, magnesium stearate, lactose, glyceryl behenate, sodium stearyl fumarate (PRUV™) and/or croscarmellose sodium. In particular, and by way of example, the present invention contemplates the following three solid ramipril formula compositions in % w/w, wherein the coating or the ramipril particles is a HPMC (Methocel E5 Prem LV) spray coating having a thickness on the order of between about 0.1 microns and 0.5 microns and being formed with about 10% solids. The spray coat has a total polymer content of from about 5%.

| | Formula | | | |
|---|---|---|---|---|
| | Compositions | I | II | III |
| (a) | coated ramipril | about 2.98% | about 2.98% | about 1.49% |
| | particles (milled) | | | |
| (b) | Prosolv ® SMCC | about 93.02% | about 94.92% | about 92.41% |
| | 50 | | | |
| (c) | glyceryl behenate | about 2.0% | — | about 4.0% |
| (d) | PRUV ™ | — | about 0.1% | about 0.1% |
| (d) | croscarmellose | about 2.0% | about 2.0% | about 2.0% |
| | sodium | | | |

As indicated above, the stabilized ramipril pharmaceutical compositions of the present invention can be administered orally or enterally to the subjects. This can be accomplished, for example, by administering to the subject a solid or liquid oral dosage form by mouth or via a gastric feeding tube, a duodenal feeding tube, a nasogastric (ng) tube, a gastrostomy, or other indwelling tubes placed in the GI tract. The oral stabilized ramipril pharmaceutical compositions of the present invention are generally in the form of individualized or multi unit doses, such as tablets, caplets, powders, suspension tablets, chewable tablets, rapid melt tablets, capsules, e.g., a single or double shell gelatin capsule, tablet-filled capsules, effervescent powders, effervescent tablets, pellets, granules, liquids, solutions, or suspensions, respectively. The oral pharmaceutical compositions may contain ramipril in any therapeutically effective amount, such as from about 1 mg or less to about 100 mg or more, or preferably from about 1.25 mg to about 50 mg, or preferably from about 1.25 mg to about 20 mg. By way of example, a stabilized oral unit dose or composition of the present invention may contain ramipril in a dosage amount of about 1.25 mg, about 2.5 mg, about 5 mg, about 7.5 mg, about 10 mg, 12.5 mg, about 15 mg, about 20 mg, about 25 mg, about 30 mg, about 40 mg, about 50 mg, about 60 mg, about 70 mg, about 75 mg, about 80 mg, about 90 mg, or about 100 mg. Of course, it should be appreciated that a particular unit dosage form and amount can be selected to accommodate the desired frequency of administration used to achieve a specified daily dosage and therapeutic effect.

Consistent with the present invention, these and other dosage forms discussed herein may be administered to individuals on a regimen of one, two or more doses per day, at any time of the day or whenever needed to treat a cardiovascular disorder.

While the present invention contemplates any solid dosage form suitable for oral administration, ramipril tablets, capsules, tablet-filled capsules and caplets are especially preferred. When the stabilized ramipril compositions of the present invention are formed into tablets or caplets, it is to be understood that the tablets or caplets may be scored, and that they may be of any suitable shape and size, such as round, square, rectangular, oval, diamond, pentagon, hexagon or triangular, so long as the objectives of the present invention are not defeated. It is to be further understood that when tablet-filled capsules are selected, the tablets utilized therewith may be formed into shapes that either (a) correspond to the capsules to permit over-coating or encapsulation via the capsules or (b) readily fit inside the capsules. Of particular interest are stabilized 1.25, 2.5, 5, 10, 15 and 20 mg ramipril tablets, stabilized 1.25, 2.5, 5, 10, 15 and 20 mg ramipril caplets, stabilized 1.25, 2.5, 5, 10, 15 and 20 mg ramipril capsules and stabilized 1.25, 2.5, 5, 10, 15 and 20 mg ramipril tablet-filled capsules.

An article of manufacture, as contemplated by the present invention, comprises a container holding a pharmaceutical composition suitable for oral administration of stabilized ramipril in combination with printed labeling instructions providing a discussion of when a particular dosage form should be administered. The composition will be contained in any suitable container capable of holding and dispensing the dosage form and which will not significantly interact with the composition and will further be in physical relation with the appropriate labeling advising that a dosage form is more stable and bioavaliable with extended shelf-life. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

The examples throughout herein and that follow are provided solely to illustrate representative embodiments of the invention. Accordingly, it should be understood, that the invention is not to be limited to the specific conditions or details described in these or any other example discussed herein, and that such examples are not to be construed as limiting the scope of the invention in any way. Throughout the specification, any and all references are specifically incorporated herein by reference in their entireties.

### EXAMPLES

Ramipril

Preparation of ramipril is described in U.S. Pat. Nos. 5,061,722 and 5,403,858, which are incorporated by reference in their entireties. Briefly, cis, endo-2-azabicyclo-[3.3.0]-octane-3-carboxylic acid is reacted with benzyl alcohol and thionyl chloride to form the benzyl ester, which is then reacted with HOBr and N-(1-S-carbethoxy-3-phenylpropyl)-S-alanine to form benzyl N-(2-S-carbethoxy-3-phenylpropyl)-S-alanyl-cis, endo-2-azabicyclo-[3.3.0]-octane carboxylate. The mixture can be chromatographed or recrystallized to isolate the S,S,S and S,S,R isomers. Reduction of the L,L,L benzyl ester provides ramipril.

U.S. Pat. No. 6,407,262, also incorporated by reference herein in its entirety, provides a method for separating the diastereomeric mixtures of ramipril, the synthesis of which is also described therein. Briefly, mixtures of the benzyl diastereoisomers of ramipril are acidified in an organic solvent and allowing the desired isomer to precipitate. Ramipril is obtained by removal of the benzyl group by catalytic dehydrogenation.

Alternatively, ramipril is manufactured by and obtained from Aventis Pharma Deutschland GmbH (Frankfurt on Main, Germany).

U.S. Pat. No. 5,055,591, incorporated herein by reference in its entirety, also describes preparation of ramipril from the benzyl ester, as described above.

Ramipril may be used as its ethyl, methyl, or isopropyl ester or other diester forms or suitable derivatives, where the ester groups are readily metabolized after administration to form ramiprilat, the dicarboxylic acid, which is the active form of ramipril in vivo. Ramipril ethyl ester is preferred.

Ramipril is obtained from Aventis Pharma Deutschland GmbH (Frankfurt on Main, Germany), CAS number 87333-19-5, as a white, odorless crystal having a melting range of about 108-109° C., bulk density of about 77-125 kg/m3. The material forms a suspension in water at about pH 4.6, is soluble in methanol (about 339 g/ml at about 20° C.) and relatively insoluble in water (about 50 g/l at 20° C.). The CAS name is (2s,3aS,6aS)-1-((S)-N-((S)-1-carboxy-3-phenyl-propyl)alanyl)octahydrocyclopenta(b) pyrrole-2-carboxylic acid, 1-ethyl ester, as described above.

It is believed that ramipril isopropyl ester, methyl ester and hexahydroramipril hydrochloride are also available from Aventis Pharma Deutschland GmbH (Frankfurt on Main, Germany).

Methods of Making Individually Coated Ramipril Particles

Hydroxypropylmethyl cellulose (HPMC) (Methocel E5 Prem LV) is dissolved in about 5, 10 and 15% of the amount of ramipril in water to obtain a final dispersion of about 30% solids wt/wt using about 30% as a starting point to determine and obtain the desired viscosity. Ramipril powder is charged and dispersed into a high-shear mixer (homogenizer; Silverson, Ross, Greerco with a square hole high sheer screen or similar types). Using an appropriate spray-drying unit and technique, ramipril is spray coated to a total polymer content of about 5, 10 or 15% (wt/wt).

The same procedure may be used by replacing HPMC with polyvinylprrolidone (PVP) or with 50:50 mixtures of HPMC and PVP.

The air used in the spray-drying process should be as dry as possible. Compressed atomization air with the lowest possible dew point should be used in a two fluid nozzle set-up. The atomization air can be heated using a flow-through air heater for the fastest drying of the particles, as required by the specific molecules and process. The smallest drying zone will prevent particle agglomeration.

It is important to use the smallest possible spray nozzle, but to avoid clogging, the opening should be at least about 3 particle diameters in width. The ramipril dispersion should be homogenized long enough to obtain the smoothest possible suspension with little to no powder agglomerates. The homogenization can be checked visually with a spatula or similar device. Additionally, the dispersion should be viscous enough to suspend the particles without immediate separation, but fluid enough to allow pumping to the fluid-bed nozzle(s) with minimal setting. Adequate atomization must be allowed to achieve individual coated particles. It is important that the particles not be allowed to settle in the hoses, spray arm, or the nozzle in order to avoid clogging.

The following outlines an exemplary procedure:

1. Delump ramipril by passing it through a 20-mesh screen into an appropriately sized labeled container.

2. Add about 35% of the total purified water to an appropriately sized container and mix with an overhead mixer to provide sufficient agitation and shear to produce a vortex without introducing excessive air into the liquid.

3. Slowly charge the polymer (HPMC and/or PVP) into the purified water with continuous mixing (adjust the mixer speed as required to maintain a sufficient vortex without introducing excessive air into the liquid).

4. Mix Step 3 for a minimum of about 30 minutes or until the polymer is completely dissolved.

5. Add the delumped API from Step 1 to the remaining about 65% of the total purified water with continuous mixing using an appropriate mixer/homogenizer (using a high shear unit to produce a uniform/smooth dispersion). Mix the dispersion for a minimum of about 15 minutes or until the API is uniformly dispersed with no visible agglomerates.

6. Add the polymer solution from Step 4 to the API dispersion in Step 5 above with continuous mixing using the appropriate mixer/homogenizer from Step 5 above. (Rinse the polymer solution container as required with a small portion of purified water to achieve a complete transfer). Remove the homogenizer and mix the dispersion with a high shear overhead mixer for a minimum of about 15 minutes or until the API is uniformly dispersed without introducing air into the dispersion.

7. Continue to mix the dispersion throughout the entire spray-drying process.

8. Set-up the spray drier as required (two fluid nozzle, spin plate, drying chamber, cyclone, and collection chamber). Preheat the unit to the appropriate temperature. Adjust the atomization pressure or spin plate speed.

9. Spray the dispersion using the following parameter ranges as a guide (parameters may be adjusted as required):
Inlet Temperature—about 90 to 100° C.
Outlet Temperature—about 25 to 30° C.
Atomization Air Pressure—about 1.5 to 2.0 bars
Pump Rate—as required by unit to achieve highest flow rate with adequate drying
Aspirator—about 65 to 85%

The outlet temperature may be increased to achieve complete drying and avoid agglomeration of the coated API particles, but it is critical to keep this temperature as low as possible to avoid undue degradation of the product.

10. Continue spraying the dispersion with constant mixing until it is depleted.

Tables 1 and 2 show several alternative coating formulations.

TABLE 1
Coating Formulations 30% Solids by Weight

| Material | Mg | % | Coating | Solids |
|---|---|---|---|---|
| API | 420.000 | 28.57% | 5.00% | 30.00% |
| HPMC | 21.000 | 1.43% | | |
| Water | 1029.000 | 70.00% | | |
| | 1470.000 | 100.00% | | |
| API | 420.000 | 27.27% | 10.00% | 30.00% |
| HPMC | 42.000 | 2.73% | | |
| Water | 1078.000 | 70.00% | | |
| Total | 1540.000 | 100.00% | | |
| API | 420.000 | 26.09% | 15.00% | 30.00% |
| HPMC | 63.000 | 3.91% | | |
| Water | 1127.000 | 70.00% | | |
| | 1610.000 | 100.00% | | |

TABLE 2
Coating Formulations 50% Solids by Weight

| Material | Mg | % | Coating | Solids |
|---|---|---|---|---|
| API | 420.000 | 47.62% | 5.00% | 50.00% |
| HPMC | 21.000 | 2.38% | | |
| Water | 441.000 | 50.00% | | |
| Total | 882.000 | 100.00% | | |
| API | 420.000 | 45.45% | 10.00% | 50.00% |
| HPMC | 42.000 | 4.55% | | |
| Water | 462.000 | 50.00% | | |
| Total | 924.000 | 100.00% | | |
| API | 420.000 | 43.48% | 15.00% | 50.00% |
| HPMC | 63.000 | 6.52% | | |
| Water | 483.000 | 50.00% | | |
| Total | 966.000 | 100.00% | | |

Particle Size Analysis and SEM Images of Ramipril Spray-Dried Preparations

The samples are dried powders comprising dried about 10% solids, about 5% coating (wet) identified as Batch N1440-19.

A portion of the powder sample is suspended in silicone oil on a microscope slide and a cover glass applied. The sample is viewed with a light microscope at a magnification of 100×. The microscope slide preparation is scanned using a mechanical stage and is sized using a calibrated eyepiece reticle. A minimum of 1000 particles are counted and the results are placed in the following range categories: about 0-20 μm; about >50-100 μm; about >100-150 μm; and about >150 μm. The results of the particle size distribution analysis are summarized in Table 3. Large crystal agglomerates as large as about 537.5 μm are observed. Photomicrographs of the large agglomerates from two different fields of view are taken.

A portion of the powder is sprinkled onto a conductive carbon tape tab which is attached to an aluminum substrate. An about 100 Angstrom coating of gold/palladium is applied to the sample, providing the particles with a conductive surface. Imaging of the particles is performed in a JEOL 6301 field emission scanning electron microscope. Images of some of the larger crystal agglomerates are taken (e.g., see FIG. 4A). The images of three different groups of particles are taken at the following magnifications: about ×100, about ×300, and about ×750. FIGS. 1-3 illustrate the crystals of ramipril from the spray-dried preparations.

TABLE 3
Microscopic Particle Size Distribution of Ramipril Spray-dried
10% Solids, 5% Coating Wet, Batch N1440-19

| | 0-20 | >20-50 | >50-100 | >100-150 | | |
|---|---|---|---|---|---|---|
| | μm | μm | μm | μm | >150 μm | Total |
| # Particles | 852 | 155 | 12 | 4 | | 1034 |
| (%) | (82.5%) | (15%) | (1.2%) | (0.4%) | | |

A Process for Preparing Spray-Dried Ramipril

Numerous trials are conducted with about 30% solids/5% coating; about 10% solids/5% coating and about 20% solids/5% coating. About 200 g ramipril suspension formulations are employed.

A 48 kHz Sono-tek ultrasonic nozzle equipped with a Glatt Spray Dryer is used. The suspension is most effectively atomized with about 10% solids/5% coating formula, but did provide fair results with the about 20% solids/5% coating formula. Spray rate and atomization powder are adjusted accordingly to achieve a fine mist from the nozzle.

Spray-drying tests are performed on about 1-kg ramipril suspensions and are evaluated using the Glatt Passive Flow Spray Dryer. The rate is first set at about 5-6 g/min with an inlet temperature of about 100° C. There is poor airflow, resulting in poor distribution of the material on the collection pan, likely due to too high a spray rate at the set temperature. After completion of the spraying, the material remains in the heated chamber to further dry the material. As a result, the material overheats and becomes slightly scorched and discolored prior to removal from the chamber.

Drying of the material is improved by decreasing the spray rate to about 4 g/min. Fluctuations in the spray rate are observed after about 12 hr of spraying. The suspension, is warmed as a result of contact with the heated nozzle, is gradually clogging the flow from the nozzle. Although the nozzle is back flushed with water to remove any accumulated material, the flow rate is continued to decrease until it is apparent the nozzle again becomes clogged. After about 2.5 hrs. with about a third of remaining suspension, the process is aborted and the material collected is dried overnight in the pan at ambient temperature.

To ensure deagglomeration of the material and possible particle reduction, the about 10% solids dispersion is homogenized immediately prior to spraying for a total of about 20 minutes beyond the mixing time that is previously used. Homogenization is performed using an Omni Homogenizer 5000 equipped with a 20 mm generator probe at a speed setting of “3”. Homogenization is attempted with a 35 mm generator, but mixing is too vigorous with this size of probe for the IL volume of suspension.

Steady atomization of the dispersion is maintained throughout a subsequent trial using a spray rate of about 3-4 g/min at an inlet temperature of about 1050° C. However, the rate of drying is not adequate. This results in the collection of material on the pan, which appears to be dry around the perimeter, with a sizable central wet portion. The material collected in the pan is allowed to dry overnight at ambient temperature. The following day the material is removed from the pan and divided into “wet” and “dry” portions based on visual appearance of the boundary, then it is placed in separate dishes. This material is placed in the Laminar Flow Hood to complete drying. The total room temperature drying required about 25 hrs. for the “dry” portion and about 42.5 hrs. for the “wet” portion.

To maintain the temperature at about <100° C., the flow rate of the suspension can be further decreased to achieve adequate drying. An additional peristaltic pump with smaller ID tubing can be used to allow slower flow rates, thus allowing for improved drying. The reduced flow can be set at about 2 g/min.

The spray-dried material from the about 20% solids/5% coating and about 10% solids/5% coating (“wet” and “dry” portions) is screened through #20 mesh and is stored protected from light.

Spray-Drying Evaluation of 10% Solids/5% Coating

A batch is manufactured as a spray-dried ramipril to evaluate the effect on DKP growth by decreasing the spray rate. The expectations from this batch are to further improve the drying process by reducing the droplet size. After one failed manufacturing attempt due to a malfunction of equipment, a new generator is obtained from Sono-Tek, for the 48 hz nozzle, to ensure greater control of the atomization pressure.

The composition of the coating dispersion for about 10% solids/about 5% coating is listed below in Table 4:

TABLE 4

| Ingredients | % w/w | Batch weight (g) |
|---|---|---|
| Ramipril | about 9.5 | about 95 |
| Methocel E5 PREM LV | about 0.5 | about 5 |
| Purified Water for HPMC solution | about 31.5 | about 315 |
| Purified Water for API solution | about 58.5 | about 585 |
| Total | about 100.0 | about 100.0 |

A description of the procedure for preparation of about 1 L batch of the spray dispersion is listed below with actual mix times and temperatures stated in parentheses ( ):

A. HPMC Solution
a. Pass Methocel (about 5 g) through a 20-mesh screen and slowly incorporate into purified water (about 315 g) while stirring with a lab mixer. (Time of addition=about 12 min.).
b. Apply heat as necessary and stir until dissolved. (Total mixing time=about 50 min. final temp=about 42° C.).
c. Cover and set aside.

B. API Dispersion
a. Pass Ramipril (about 95 g) through a 20-mesh screen and slowly add to purified water (about 585 g), using a planetary mixer. (time of addition=about 2 minutes and 37 sec.)
b. Stir for no longer than about 15 minutes after addition of API to achieve uniform dispersion. (Total mixing time=about 19 min. and 30 sec.).

C. Spray-Drying Dispersion (A+B)
a. Slowly add HPMC solution to API dispersion and mix for about 15 min. using a planetary mixer (Total mixing time=about 15 min.).
b. Transfer into HPMC container & maintain slow stirring with a stir bar until spray-drying process.

The spray-drying process is conducted on the following day using the Glatt Lab Spray unit equipped with about 48 Hz ultrasonic spray nozzle. The following parameters are selected to initiate the run:
a. set air temperature=about 100° C.;
b. spray rate=about 2 g/min; and
c. atomization pressure=about 5.5 (about first 15 mins)−about >7.6 watts

The spray dispersion is pumped through a peristaltic pump into the spray nozzle and is atomized by an external generator. Prior to starting the process, the spray dispersion is homogenized to de-agglomerate any particles. A perforated removable collection pan lined with white pharmaceutical grade paper is placed in the chamber.

When the process begins, the droplets appear to be dry as they fall to the collection pan, but condensation within the chamber soon causes the material to dry less efficiently. Inspection of the spray-dried API collected on the pan reveals a concentration of moist particles at the center. The tray paper is replaced periodically to avoid an excess accumulation of wet API. Attempts are made to optimize the drying rate by adjustment of the stack height, to increase the velocity of air inside the unit. Also, gradual incremental increases in the inlet set temperature from about 100° C. to about 125° C. are made to improve the drying capacity of the air. It is determined that about 120° C. is the maximum temperature allowable to avoid visible discoloration of the paper/material and possible further product degradation. The spray atomization is also decreased to about 6.6 watts about midway through the run to maintain a consistent spray mist.

Even with the adjustments, optimal drying conditions, yielding completely dried material on the collection pan, are never achieved. As the tray papers are removed from the chamber approximately every hour, they are placed in a HEPA filtered flow hood overnight to complete drying.

After drying, all of the material collected is combined as one sample and passed through a 20 mesh screen. The batch yields about 59.2% spray-dried API (58.0 g). A sample of the finished product is submitted to analytical for assay, related substances, and water content testing. The results are shown below in Table 5:

TABLE 5

| Test | Result |
|---|---|
| Ramipril Assay | about 53.35% |
| Ramipril Degradant | about 33.96% |
| Products (including | |
| ramipril DKP) | |
| Water Content | about 0.7% |

The HPMC concentration and other degradants are not quantitated. Since the product degradation is so high, it is decided to not proceed with photomicroscopy or further batch manufacture.

It is noted that an earlier experiment produced spray-dried material (wet) with a much lower level of DKP. In this experiment, the wet mass is separated from the dried material, subsequently air dried, and then tested for assay and DKP with results of about 97.7% and about 4.2%, respectively. For B0001F1A, the increase in temperature during spray-drying may have contributed to an increase in degradation. The ‘wet’ spray-dried API collected in the initial experiments is visibly more moist than the ‘wet’ material from B0001F1A due to a higher spray rate at a lower temperature.

Based on the operational design of the Glatt unit, it may be difficult to achieve material truly representative of a spray-dried API. It is observed that the air turbulence within the chamber of the Glatt is much lower than that observed in other systems.

Spray-Drying Evaluation of 10% Solids/5% Coating

A batch is prepared as a ramipril/HPMC dispersion to evaluate the spray-drying process with the Buchi B-290 Minispray Dryer.

The composition of the coating dispersion for about 10% solids/about 5% coating is listed below in Table 6:

TABLE 6
About 10% Solids/5% Coating Ramipril-HPMC Dispersion

| Ingredients | % w/w | Batch weight (g) |
|---|---|---|
| Ramipril | about 9.5 | about 95 |
| Methocel E5 PREM LV | about 0.5 | about 5 |
| Purified Water for HPMC | about 31.5 | about 315 |
| solution | | |
| Purified Water for API | about 58.5 | about 585 |
| solution | | |
| Total | about 100.0 | about 1000 |

A description of the procedure for preparation of a 1 L batch of the spray dispersion is listed below with actual mix times and temperatures stated in parentheses ( ):

A. HPMC Solution
a. Pass Methocel (about 5 g) through a 20-mesh screen and slowly incorporate into purified water (about 315 g) while stirring with a lab mixer. (Time of addition=about 22 min.).
b. Stir until dissolved. (Total mixing time=about 45 min.; final temp=about 24.4° C.).
c. Cover and set aside.

B. API Dispersion
a. Pass Ramipril (about 95 g) through a 20-mesh screen and slowly add to purified water (about 585 g), using a planetary mixer (time of addition=about 5 min.)
b. Stir for NLT about 15 min. after addition of API to achieve uniform dispersion. (Total mixing time=about 15 min.).

C. Spray-Drying Dispersion (A+B)
a. Slowly add HPMC solution to API dispersion and mix for about 15 min. using a planetary mixer (Total mixing time=15 min.).
b. Transfer into HPMC container & maintain slow stirring with a stir bar until spray-drying process.

During the holding period, the dispersion is kept tightly covered with continual stirring. Prior to spray-drying, the dispersion does not appear to have settled or agglomerated.

The parameters to be used for the three different trials are listed in Table 7, followed by the % yield that is obtained from each trial in Table 8.

TABLE 7
Buchi B-290 Mini Sprayer Dryer Spray-drying Parameters

| Parameters | Trial A | Trial B | Trial C |
|---|---|---|---|
| Preset Inlet Temperature (° C.) | about 100 | about 125 | about |
| | | | 150 to 140 |
| Actual Inlet Temperature (° C.) | about 94 | about 120 | about 140 |
| Outlet Temperature (° C.) | about 59 | about 50-54 | about 54 |
| Spray Rate (%) | about 25 | about 25 | about 25 |
| | (8 g/min) | | |
| Aspirator Power (%) | about 100 | about 100 | about |
| | (about | | 100 to 90 |
| | 35-40 m³h | | |
| | air flow | | |
| Air Pressure (psi) | about 85 | about 85 | about 85 |
| Spray Flow Meter (mm) | about 30-40 | about 30-40 | about |
| | | | 30-40 |
| Total Time of Spraying | about 27:45 | about 26:59 | about |
| | | | 25:01 |

TABLE 8
Percent Yield of Spray-Dried Material

| Results | Trial A | Trial B | Trial C |
|---|---|---|---|
| Amount of Dispersion Used (g) | about 197.80 | about 190.66 | about |
| | | | 176.8 |
| Amount of Spray-Dried Powder | about 10.22 | about 9.42 | about 3.03 |
| (g) | | | |
| Theoretical Amt. Of Solids (g) | about 19.78 | about 19.07 | about |
| | | | 17.68 |
| % Yield of Solids | about 51.7 | about 49.4 | about 17.1 |

Trials A & B proceeded without incident, however, at the start-up of Trial C, it is observed that powder is collecting on the inside walls of the cyclone. Within about 15 minutes into the spray cycle, it is necessary to decrease the temperature from 150° C. to 140° C. and lower the aspirator rate from 100% to 90% to avoid over drying the material and move it through the cyclone into the collection vessel. As a result, the yield is very low for this run due to the loss of material during spraying. The material collected from all of the spray-drying trials is very light and powdery similar to ramipril, rather than granular like the GeCoated ramipril.

Samples of the spray-dried material from all three batches are submitted to analytical for assay, related substances, and water content. Those results along with the Glatt trials are shown in Table 9.

TABLE 9
Assay, DKP, Water Content of Spray-Dried Material

| | Ramipril | Buchi | Buchi | Buchi | Glatt | Glatt |
|---|---|---|---|---|---|---|
| | Control | Trial A | Trial B | Trial C | N1440-19/dry | N144019/wet |
| Test | Batch A080 | (100° C.) | (125° C.) | (140° C.) | (105° C.) | (105° C.) |
| Assay(% w/w) | about 100.5 | about 94.9 | about 95.1 | about 94.7 | about 76.9 | about 97.7 |
| DKP (% w/w) | about 0.2 | about 0.3 | about 0.3 | about 0.6 | about 21.4 | about 4.2 |
| Water (% w/w) | about 0.2 | about 1.2 | about 0.7 | about 0.6 | about 0.8 | about 0.6 |

Compared to work that is performed with the Glatt Lab Sprayer, the Buchi spray dryer process shows a dramatic improvement in reducing DKP growth of the finished product. The 125° C. material appears to provide the best results based on the balance of the lowest degradant and water content compared to the control sample. SEM images are taken of Samples from trials A and B.

Spray-Drying Evaluation of about 30% Solids/5% Coating and 30% Solids/15% Coating

Batch B0003F2 is prepared as a Ramipril/HPMC dispersion to evaluate the spray-drying process with a higher percentage of solids to increase the yield of API. With successful processing of about 30% solids content, it is decided to increase the HPMC coating. Batch B0004F3 is prepared as a Ramipril/HPMC dispersion to evaluate the spray-drying process with about 15% coating.

The composition of the coating dispersions for each batch is listed below in Table 10:

TABLE 10
Formulation Matrix

| | BATCH | | | |
|---|---|---|---|---|
| | B0003F2 | | B0004F3 | |
| | about | | about | |
| | 30% solids/5% coating | | 30% solids/15% coating | |
| | | Batch | | Batch |
| Ingredients | % w/w | Weight (g) | % w/w | Weight (g) |
| Ramipril | about 28.57 | about 142.85 | about 26.09 | about 130.45 |
| Methocel E5 | about 1.43 | about 7.15 | about 3.91 | about 19.55 |
| PREM LV | | | | |
| Purified Water | about 24.50 | about 122.50 | about 15.00 | about 75.00 |
| for HPMC | | | | |
| solution | | | | |
| Purified Water | about 45.50 | about 227.50 | about 55.00 | about 275.00 |
| for API | | | | |
| suspension | | | | |
| Total | about 100.0 | about 500.00 | about 100.00 | about 500.00 |

A. HPMC Solution
a. Pass Methocel through a 20-mesh screen and slowly incorporate into purified water while stirring with a lab mixer.
b. Stir until dissolved.
c. Cover and set aside.

B. API Suspension
a. Pass Ramipril through a 20-mesh screen and slowly add to purified water, using a planetary mixer.
b. Stir for no less than about 15 minutes after addition of API to achieve homogenous suspension.

C. Spray-Drying Dispersion (A+B)
a. Slowly add HPMC solution to API suspension and mix for about 15 minutes using a planetary mixer.
b. Transfer into HPMC container & maintain slow stirring with a stir bar until spray-drying process is completed.

D. Spray-Drying—Trials 1A/2A@ about 100° C. and 1B/2B@ about 100° C.
a. Homogenize the dispersion for about 3-5 minutes using the Omni Homogenizer with the 35 mm probe.
b. Divide the dispersion into two portions for each temperature trial.
c. Set-up Buchi spray dryer with a 1.5 mm nozzle to the specified parameters for about 1001° C. trial.
d. Process the material for about 25 minutes.
e. Allow the equipment to cool down for about 30 minutes.
f. Process remaining material.
g. Clean equipment and repeat steps c-f for about 125° C. trial.

During the holding period, the dispersion is kept tightly covered and continually stirred. Prior to spray-drying, the dispersion does not appear to have settled or agglomerated. The water portion used in the preparation of the API suspension is increased in B0004F3 from B0003F2 to better incorporate the higher concentration of Ramipril in the about 30% solids formula.

The parameters used for the four different trials of the two batches (B0003F2-1 A/1 B and B0004F3-2A/2B), are listed in Table 11, followed by the percent yield obtained from each trial in Table 12.

TABLE 11
Buchi B-290 Mini Sprayer Dryer Spray-drying Parameters

| | B0003F2 | | B0004F3 | |
|---|---|---|---|---|
| | Step D | Step E | Step D | Step E |
| Parameters | Trial 1A | Trial 1B | Trial 2A | Trial 2B |
| Preset Inlet Temperature (° C.) | about 100 | about 125 | about 100 | about 125 |
| Outlet Temperature (° C.) | about 54-57 | about 64-67 | about 48-55 | about 56-63 |
| Spray Rate (%) | about 25→20 | about 25→20 | about 20 | about 20 |
| | about 8→6 g/min) | | | |
| Aspirator Power (%) | about 100→95 | about 100→95 | about 100 | about 100 |
| | (about 40→35³/n | | | |
| | air flow) | | | |
| Air Pressure (psi) | about 85 | about 85 | about 85 | about 85 |
| Nozzle Cleaner | about 5 | about 5 | about 5 | about 5 |
| Spray Flow Meter (mm) | about 30-40→45 | about 45 | about 45 | about 45 |
| Total Time of Spraying | about 40:49 | about 44:08 | about 44:44 | about 41:32 |
| (minutess:seconds) | | | | |

NB Ref: N1313-70 and N1488-84, L1319-27

TABLE 12
Percent Yield of Spray-Dried Material

| | B0003F2 | | B0004F3 | |
|---|---|---|---|---|
| | Step D | Step E | Step D | Step E |
| Results | Trial 1A | Trial 1B | Trial 2A | Trial 2B |
| Amount of | about 232.18 | about 241.27 | about 239.59 | about 224.00 |
| Dispersion | | | | |
| Used (g) | | | | |
| Amount of | about 42.55 | about 48.41 | about 33.10 | about 38.01 |
| Spray-Dried | | | | |
| Powder (g) | | | | |
| Theoretical | about 69.65 | about 72.38 | about 71.88 | about 67.20 |
| Amt. Of Solids | | | | |
| (g) | | | | |
| Yield of Solids | about 61.09 | about 66.88 | about 46.05 | about 56.56 |

Due to the higher solids content of the batches, the pump rate is decreased in Trial 1A from 25% to 20%. The setting is maintained for all remaining trials. Within a few minutes into the spray cycles, the spray cyclone becomes filmed with a layer of product. To reduce build-up in the cyclone, avoid over drying the material, and move it through the cyclone into the collection vessel, it is necessary to lower the aspirator rate from 100% to 95% (as noted in Table 11). The material collected from all of the spray-drying trials is very light and powdery similar to Ramipril, rather than granular like the GeCoated Ramipril.

Samples of the spray-dried material from both batches were submitted to analytical for assay, related substances, and water content. Those results are shown in Table 13, along with the previous trials (B0002F1A—trials A & B).

TABLE 13
Assay, Degradants: DKP & Ramiprilat, and Water Content of Spray-Dried Ramipril

| | | | | 30% solids/ | | 30% solids/ | |
|---|---|---|---|---|---|---|---|
| | | | | 5% coating | | 15% coating | |
| | Control | 10% solids/5% coating | | B0003F2 | B0003F2 | B0004F3 | B0004F3 |
| | Ramipril | B0002F1A | B0002F1A | (Step D) | (Step E) | (Step D) | (Step E) |
| | Batch | Trial A | Trial B | Trial 1 A | Trial 1B | Trial 1B | Trial 2B |
| Test | A080 | 100° C. | 125° C. | 100° C. | 125° C. | 100° C. | 125° C. |
| Assay | about | about 94.9 | about 95.1 | about 94.9 | about 96.7 | about 88.1 | about 88.3 |
| (% w/w) | 100.5 | | | | | | |
| Theoretical | about N/A | about 95 | 95 | about 95.2 | 95 | about 85.0 | 85.0 |
| (% w/w)¹ | | | | | | | |
| DKP | about 0.2 | about 0.3* | about 0.3* | about 0.13 | about 0.19 | about 0.10 | about 0.09 |
| (% wlw)² | | | | | | | |
| Ramiprilat | about 0.06 | about | about | about 0.07 | about 0.08 | about 0.09 | about 0.09 |
| (% wlw)³ | | 0.13* | 0.14* | | | | |
| Water | about 0.2 | about 1.2 | about 0.7 | about 0.9 | about 0.8 | about 1.1 | about 1.1 |
| (% wlw)⁴ | | | | | | | |

*Note:
Higher levels of degradants may be related to elapsed time from dispersion manufacture to spray-drying (total 7 days).

The elapsed time of 7 days from manufacture of the dispersion to the actual spray-drying process may account for increases in DKP and ramiprilat degradants in the first set of experiments (B0002F1A—Trials A&B). Both B0003F2 and B0004F3 are processed within 1 day of preparation of the spray dispersion and spray-drying. Increasing the concentration of HPMC in the about 30% solids/15% coating formula (B004F3) does show improvement in slowing down degradation

Table 14 shows results of processing tests for spray-dried samples for (1) about 20% solids/5% coating; (2) about 10% solids/5% coating (dry); and (3) about 10% solids/5% coating (wet).

TABLE 14

| | | | 10% solids |
|---|---|---|---|
| | 20% solids | 10% solids | 5% coating (wet |
| | 5% coating | 5% coating (dry) | coating) |
| Test | (Ref: N1440-14) | (Ref: N1440-19) | (Ref: N1440-19) |
| Assay (%) | 30.62 | 76.92 | 97.70 |
| Related | 69.54 | 21.38 | 4.21 |
| Substances | | | |
| (% DKP) | | | |
| Water Content | 0.69 | 0.81 | 0.59 |
| (%) | | | |

Coating Thickness

Individually coated ramipril particles were coated with dispersions containing 25-30% solids wherein the amount of coat forming material was between 20% to 30% by weight of ramipril. After the individually coated ramirpil particles were formed they were formulated into tablets and the thicknesses of the individually coated ramipril particles were measured. Table 15 shows the spray coating formulation and the coating thickness of the individually coated ramipril particles.

TABLE 15

| | HPMC (% of | Total Solids | Coating |
|---|---|---|---|
| | total weight of | in | Thickness |
| Batch No. | ramipril) | Despersion | (inches) |
| B0031F26 | 30% | 25% | 0.006-0.008 in |
| B0032F27 | 25% | 30% | 0.004-0.010 in |
| B0033F28 | 20% | 30% | 0.011-0.023 in |

The thickness of the coatings were measured by first sputter coating the tablets with a thin layer of gold (20-50 nm) and then shearing off a side of the tablet to exposing individually coated ramipril particles. Measurements were taken with an electron microscope.

Preparation of Ramipril Tablets

A process for preparation of ramipril tablets is described. This process can be scaled, for example, to about 6 kg, in a 16-quart V-shell PK blender, and larger as needed. Tablets can be produced with a Fette P1200 24-station press, or similar equipment.

Prosolve® SMCC 50 is pre-blended with the coated ramipril prepared as in Example 1, milled with glyceryl behenate, PRUV™ and croscarmellose sodium in a 16-quart V-shell blender for about 20 min, then mill-blended through Quadro Comil. The mixture is transferred to a 16-quart container and mixed for about 8 minutes, then compressed on a Stokes B2 tablet press, tooled with 16 stations with ¼′ standard concave (about 100 mg tablet weight) or 5/16″ standard concave (about 200 mg tablet weight) double-sided debossed tooling at about 48 rpm.

Stability of API ramipril co-milled to about 40 and 60 mesh and about 60 mesh, about 6 kg batch size shown in Table 16.

TABLE 16
Ramipril Tablets Stability
API Co-milled ˜40mesh

| | | % LC | | | | | |
|---|---|---|---|---|---|---|---|
| Lot # | Strength | Initial | 2 wk 40/75 | 4 wk 40/75 | 8 wk 40/75 | 12 wk 40/75 | 12 wk RT |
| 58F60A | 1.25 mg | 107.4 | 108.7 | 108 | 104.6 | 104.5 | 108.5 |
| 59F61A | 1.25 mg | 104.6 | 108.2 | 107.3 | 106.6 | 104.1 | 107.9 |

API Co-milled ˜60mesh

| | | % LC | | | | | | |
|---|---|---|---|---|---|---|---|---|
| Lot # | Strength | Initial | 2 wk 40/75 | 4 wk 40/75 | 8 wk 40/75 | 8 wk RT | 12 wk 40°/75 | 12 wk RT |
| 73F74A | 1.25 mg | 104.0 | 102.0 | 103.4 | 101.6 | 102.8 | 101.2 | 103.0 |
| 74F75A | 1.25 mg | 104.4 | 101.7 | 103.23 | 99.7 | 102.8 | 101.1 | 104.3 |

API Co-milled ˜60mesh 6 kg Batch Size

| | Strength | % LC | | | | |
|---|---|---|---|---|---|---|
| Lot # | Initial | 2 wk 40/75 | 4 wk 40/75 | 8 wk 40/75 | 4 wk RT | 8 wk RT |
| 76F74A | 1.25 mg | 104.4 | 102.7 | 102.6 | 100.4 | 104.4 |

Table 17 is stability of API co-milled tablets to about 60 mesh from a fluid bed granulation showing both GeCoated agglomerate ramipril and neat API ramipril

TABLE 17
API Co-milled ˜60mesh Fluidbed Granulation
(GeCoated and neat API)

| | | % LC | | | | | % DKP | | | | | CU |
|---|---|---|---|---|---|---|---|---|---|---|---|---|
| | | | 2 wk | 4 wk | 8 wk | 12 wk | | 2 wk | 4 wk | 8 wk | 12 wk | % LC/ |
| Lot # | Strength | Initial | 40/75 | 40/75 | 40/75 | 4075 | Initial | 40/75 | 40/75 | 40/75 | 40/75 | % rsd |
| 69F70A | 1.25 mg | 105.0 | 104.1 | 108.4 | 105.7 | 105.6 | 0.35 | 0.61 | 0.85 | 1.24 | 1.63 | 107.9/1.6 |
| 70F71A | 1.25 mg | 94.4 | 90.0 | 90.5 | 87.6 | 84.6 | 0.20 | 0.63 | 2.39 | 5.91 | 8.04 | 93.9/0.9 |
| 71F72A | 1.25 mg | 96.4 | 97.2 | 100.2 | 99.3 | 99.4 | 0.33 | 0.62 | 0.78 | 1.10 | 1.50 | 98.5/2.5 |
| 72F73A | 1.25 mg | 97.5 | NT | NT | NT | NT | 0.24 | NT | NT | NT | NT | 100.1/4.1 |

An immediate release prescription ramipril tablet equivalent to the existing capsule dosage form is described. The objective is to have a robust form of the drug, acceptable content uniformity, and similar dissolution profiles and stability when compared with the capsule.

Formulation is tested with the following ingredients (Table 18):

TABLE 18

| Ingredients | Function | Composition (% w/w) |
|---|---|---|
| Coated API | Active | 1.49 |
| Glyceryl behenate | Co-lubricant and | 4.00 |
| | coating | |
| Prosolv ® SMCC50 | Diluent | 92.41 |
| Croscarmellose sodium | Disintegrant | 2.00 |
| PRUV ™ | Lubricant | 0.1 |

Table 19 lists the comparative characteristics of ramipril particle powder as purchased from Aventis Pharma (Frankfurt, Germany) and individually coated polymer ramipril particles in accordance with this invention.

TABLE 19

| API POWDER | COATED API POWDER |
|---|---|
| Crystalline white powder; | Nearly white granules |
| Columnar shaped crystals | |
| Density | |
| Bulk density: 0.14 g/ml | about 0.22 g/ml |
| Tapped density: 0.26 g/ml | about 0.27 g/ml |
| Carr's Index: 46.2% | about 18.5% |
| Mean particle size: 19.4 μm | about 74.7 μm |
| Particle size distribution: | |
| Range: 0.8-91.4 μm | about 3.9-876 μm** |

**Majority of particles are less than about 50 μm and they are comprised of small granules and individual crystals; particles greater than about 50 μm are made of clusters of particles caused by the inefficiency of the Glatt spray drier.

For comparison purposes, U.S. Pat. No. 5,442,008 describes large scale manufacture of ramipril 2.5 mg tablets that are prepared by compressing ramipril coated with about 6% HPMC film coating with microcrystalline cellulose, mannitol, and sodium stearylfumarate at a force of 10,000 N. Packaged tablets that are stored at about 40° C. for about 3 months, show about 0.6% breakdown to DKP and after about 12 months, about 5.97% DKP decomposition.

In another embodiment of the invention, ramipril, either in uncoated form, individually coated crystals or as a GeCoated agglomerate composition (polymer coated with HPMC), is coated with a blending compound (e.g., glyceryl behenate) before being processed into tablets. The co-milled ramipril is a suitable intermediate for use in preparing dry blend, direct compression formulations. Such compositions and methods relating to stable ramipril compositions are described in more detail in co-pending application Ser. No. ______, filed Nov. 7, 2005 (serial number not yet assigned). Other dosage forms, of course, are also suitable including, for example, those prepared by hot melt extrusion processes.

Typically, the blending compound is present in the tablet from at least about 0.1 wt %. In a specific embodiment, the blending compound is present at about 0.5 wt. % and above. In another specific embodiment, the blending compound is present at about 1.0 wt. % and above. In another specific embodiment, the blending compound is present at about 2.0 wt. % and above. In a specific and preferred embodiment, the blending compound is present at about 3.0 wt. % and above. In another specific embodiment, the blending compound is present at about 4 wt. % and above (e.g., 5 and 10 wt. %).

When glyceryl behenate is used as the blending compound, glyceryl behenate is present in the tablet from at least about 0.1 wt %. In a specific embodiment, glyceryl behenate is present at about 0.5 wt. % and above. In another specific embodiment, glyceryl behenate is present at about 1.0 wt. % and above. In another specific embodiment, glyceryl behenate is present at about 2.0 wt. % and above. In a specific and preferred embodiment, glyceryl behenate is present at about 3.0 wt. % and above. In another specific embodiment, glyceryl behenate is present at about 4 wt. % and above (e.g., 5 and 10 wt. %).

A process for preparation of ramipril tablets is described according to the mill-blended embodiment of the invention. As described in the flow chart in FIG. 12, the following outlines a typical process for preparing tablets from GeCoated ramipril according to this embodiment:

1. Pre-mill GeCoated ramipril though a 60-mesh screen

2. Preblend milled GeCoated ramipril with glyceryl behenate (Compritol 888 ATO) for 15 minutes in a blender that has been grounded to reduce electrostatic charges.

3. Add croscarmellose sodium, sodium stearyl fumerate (Pruv) and silicified microcrystalline cellulose (Prosolve SMCC) to Step 2 and mix for 20 minutes.

4. Co-mill contents of Step 3 through a 20-mesh sieve.

5. Place sieved material of Step 4 into blender and mix for an additional 8 minutes.

6. Compress Step 5 blend with tablet press (Stokes 0.25″ SC tooling embossed).

7. Package the finished tablets.

Direct compression tablets were prepared in accordance to the above. The components used in the test lots are described in detail below in Table 20.

TABLE 20
Batch B0046F50A
1.25 mg/90 mg

| Ingredients | % w/w | Mg/unit |
|---|---|---|
| GeCoated Ramipril (<150 μm) | 1.66 | 1.49 |
| Hand-screened* | | |
| Silicified Microcrystalline Cellulose | 94.34 | 84.91 |
| (Prosolv SMCC 50) | | |
| Croscarmellose Sodium (Ac-Di-Sol) | 2.0 | 1.8 |
| Glyceryl Behenate (Compritol 888 ATO) | 2.0 | 1.8 |
| Total | 100 | 90 |

*GeCoated API Assay (Comp# RM00364, Rec# 30002, Lot# 40A188) = 83.9% (Ref: Aventis COA - Batch 40A188)

Stability studies were conducted with the test lots. Room temperature and accelerated degradation conditions (40 degrees C. and 75% humidity) were used as the exposure conditions. As a reference dosage form Altace® was also evaluated. The results of the stability studies are graphically presented in FIG. 1. The results of the stability studies are graphically represented in FIG. 13. As can be seen in the graph lower levels of DKP are observed.

Tables 21-24 provide levels of DKP (DKP) observed for tablets containing 2 and 4 wt. percent of glyceryl behenate.

TABLE 21

| Fluid | | | | | | | | | | | | | | |
|---|---|---|---|---|---|---|---|---|---|---|---|---|---|---|
| Bed | | (GeCoated and neat API) | | | | | | | | | | | | |
| Gran. | | % LC | | | | | % DKP | | | | | CU | Formulation | |
| Batches | | | 2 wk | 4 wk | 8 wk | 12 wk | | 2 wk | 4 wk | 8 wk | 12 wk | % LC/ | (tablet run weight @ | |
| Lot # | Strength | Initial | 40/75 | 40/75 | 40/75 | 40/75 | Initial | 40/75 | 40/75 | 40/75 | 40/75 | % rsd | 100 mg) | Comments |
| 82F64A | 1.25 mg | 105.7 | 106.1 | 102.5 | 100.5 | 104.6 | 0.31 | 0.85 | 0.84 | 1.24 | 1.52 | 104.5/1.9 | GeCoated/Ceolus/ | 3% HPMC E3 |
| | | | | | | | | | | | | | Ac-di-sol/2% Compritol | Added as |
| 84F66A | 1.25 mg | 99.8 | 96.2 | 93.8 | 87.0 | 88.1 | 0.21 | 2.37 | 3.94 | 1.27 | 9.0 | 97.4/3.0 | Ramipril/Ceolus/ | binder |
| | | | | | | | | | | | | | Ac-di-sol/2% Compritol | |
| 86F68A | 1.25 mg | 99.4 | 98.0 | 98.2 | 97.4 | 97.9 | 0.28 | 0.55 | 0.69 | 1.17 | 1.36 | 102.1/4.5 | GeCoated/Lactose/ | 3% HPMC E3 |
| | | | | | | | | | | | | | Ac-di-sol/4% Compritol | Added as |
| 88F69A | 1.25 mg | 93.7 | 91.1 | 90.9 | 89.2 | 87.4 | 0.12 | 1.42 | 2.62 | 4.31 | 6.12 | 94.5/1.8 | Ramipril/Lactose/ | binder |
| | | | | | | | | | | | | | Ac-di-sol/4% Compritol | |

NT = Not tested
Limits - NMT 2.0% DKP

TABLE 22

| | | (API sized by Comill ˜40 mesh) | | | | | | | | | | | | | | |
|---|---|---|---|---|---|---|---|---|---|---|---|---|---|---|---|---|
| Compritol & Pruv | | % LC | | | | | | % DKP | | | | | | CU | | |
| Batches w/ Milled | | | | | | 12 | 12 | | | | | 12 | 12 | (EoR) | Formulation | |
| API | | | 2 wk | 4 wk | 8 wk | wk | wk | | 2 wk | 4 wk | 8 wk | wk | wk | % LC/ | (tablet run | |
| Lot # | Strength | Initial | 40/75 | 40/75 | 40/75 | 40/75 | RT | Initial | 40/75 | 40/75 | 40/75 | 40/75 | RT | % rsd | weight @ 100 mg) | Comments |
| 58F60A | 1.25 mg | 107.4 | 108.7 | 108 | 104.6 | 104.5 | 108.5 | D.29 | 0.54 | 0.91 | 1.86 | 2.30 | 0.40 | 103.7/ | GeCoated/Prosolve/ | Preblend w/ |
| | | | | | | | | | | | | | | 2.6 | Ac-di-sol/4% | Compritol & |
| | | | | | | | | | | | | | | | Compritol/0.1% | Pruv |
| | | | | | | | | | | | | | | | Pruv | |
| 59F61A | 1.25 mg | 104.6 | 108.2 | 107.3 | 106.6 | 104.1 | 107.9 | D.28 | 0.57 | 0.99 | 1.88 | 2.66 | 0.42 | 103.1/ | GeCoated/Prosolve/ | Preblend w/ |
| | | | | | | | | | | | | | | 2.6 | Ac-di-sol/2% | Pruv |
| | | | | | | | | | | | | | | | Compritol/0.1% | |
| | | | | | | | | | | | | | | | Pruv | |
| 60F62A | 1.25 mg | 104.5 | 103.1 | 104.5 | 102.0 | NT | NT | D.27 | 0.55 | 0.92 | 1.84 | NT | NT | 102.8/ | GeCoated/Prosolve/ | No Preblend |
| | | | | | | | | | | | | | | 3.1 | Ac-di-sol/4% | |
| | | | | | | | | | | | | | | | Compritol | |
| 61F63A | 1.25 mg | 106.2 | 103.8 | 105.6 | 99.1 | NT | NT | D.27 | 0.54 | 0.96 | 1.86 | NT | NT | 100.0/ | GeCoated/Prosolve/ | Preblend w/ |
| | | | | | | | | | | | | | | 2.7 | Ac-di-sol/(0.1% | Pruv - some |
| | | | | | | | | | | | | | | | Pruv) | picking |
| | | | | | | | | | | | | | | | | to push tips |

TABLE 23

| Compritol & Pruv | | (API sized by Comill ˜40 mesh) | | | | | | | | | | | | | |
|---|---|---|---|---|---|---|---|---|---|---|---|---|---|---|---|
| Batches w/ Milled | | % LC | | | | | | | % DKP | | | | | | |
| API | | | 2 wk | 4 wk | 8 wk | | 12 wk | 12 wk | | 2 wk | 4 wk | 8 wk | 12 wk | 8 wk | |
| Lot # | Strength | Initial | 40/75 | 40/75 | 40/75 | 8 wk RT | 40/75 | RT | Initial | 40/75 | 40/75 | 40/75 | 40/75 | RT | 12 wk RT |
| 73F74A | 1.25 mg | 104.0 | 102.0 | 103.4 | 101.6 | 102.8 | 101.2 | 103.0 | 0.31 | 0.64 | 1.08 | 1.87 | 2.79 | 0.35 | 0.41 |
| 74F75A | 1.25 mg | 104.4 | 101.7 | 103.2 | 99.7 | 102.8 | 101.1 | 104.3 | 0.33 | 0.67 | 1.19 | 2.22 | 3.1 | 0.37 | 0.42 |
| 75F76A | 1.25 mg | 103.9 | 100.8 | 102.4 | 102.2 | NT | 102.2 | NT | 0.31 | 0.63 | 0.98 | 1.65 | 2.48 | NT | NT |

| Compritol & Pruv | | | | |
|---|---|---|---|---|
| Batches w/ Milled | | CU (EoR) | | |
| API | | % LC/ | Formulation | |
| Lot # | Strength | % rsd | (tablet run weight @ 100 mg) | Comments |
| 73F74A | 1.25 mg | 104.7/1.5 | GeCoated/Prosolve/ | Preblend w/ |
| | | | Ac-di-sol/4% Compritol/ | Compritol & Pruv |
| | | | 0.1% Pruv | |
| 74F75A | 1.25 mg | 103.3/2.2 | GeCoated/Prosolve/ | Preblend w/ Pruv |
| | | | Ac-di-sol/2% Compritol/ | |
| | | | 0.1% Pruv | |
| 75F76A | 1.25 mg | 101.3/2.8 | GeCoated/Prosolve/ | No Preblend |
| | | | Ac-di-sol/4% Compritol | |

TABLE 24

| | | (GeCoated and neat API) | | | | | | | | | | | | |
|---|---|---|---|---|---|---|---|---|---|---|---|---|---|---|
| Fluid Bed | | % LC | | | | | % DKP | | | | | CU | Formulation | |
| Gran. Batches | | | 2 wk | 4 wk | 8 wk | 12 wk | | 2 wk | 4 wk | 8 wk | 12 wk | % LC/ | (tablet run weight @ | |
| Lot # | Strength | Initial | 40/75 | 40/75 | 40/75 | 40/75 | Initial | 40/75 | 40/75 | 40/75 | 40/75 | % rsd | 100 mg) | Comments |
| 89F70A | 1.25 mg | 105.0 | 104.1 | 108.4 | 105.7 | 105.6 | 0.35 | 0.61 | 0.85 | 1.24 | 1.63 | 107.9/1.6 | GeCoated/Ceolus-Lac/ | 3% HPMC E3 |
| | | | | | | | | | | | | | Ac-di-sol/2% Compritol | Added as |
| | | | | | | | | | | | | | | binder |
| 70F71A | 1.25 mg | 94.4 | 90.0 | 90.5 | 87.6 | 84.6 | 0.20 | 0.63 | 2.90 | 5.91 | 8.04 | 93.9/0.9 | Ramipril/Ceolus-Lac/ | |
| | | | | | | | | | | | | | Ac-di-sol/2% Compritol | |
| 71F72A | 1.25 mg | 96.4 | 97.2 | 100.2 | 99.3 | 99.4 | 0.33 | 0.62 | 0.78 | 1.10 | 1.50 | 98.5/2.5 | GeCoated/Prosolve/ | 3% HPMC E3 |
| | | | | | | | | | | | | | Ac-di-sol/4% Compritol | Added as |
| | | | | | | | | | | | | | | binder |
| 72F73A | 1.25 mg | 97.5 | NT | NT | NT | NT | 0.24 | NT | NT | NT | NT | 100.1/4.1 | Ramipril/Prosolve/ | |
| | | | | | | | | | | | | | Ac-di-sol/4% Compritol | |

2.5 mg strength tablets were also made from the spray-dry batches B0036F1, B0037F2 and B0038F33, wherein the individually coated ramipril particles have a thicker coating. Table 25 shows the stability results.

TABLE 25

| | Batch | | |
|---|---|---|---|
| | B0036F1 | B0037F2 | B0038F33 |
| % DKP | | | |
| Initial | 0.27 | 0.24 | 0.23 |
| 2 weeks | 0.85 | 0.75 | 0.83 |
| % Ramiprilat | | | |
| Initial | ND | ND | ND |
| 2 weeks | ND | ND | ND |
| % Other | | | |
| Degrdants: | | | |
| Ramipril methyl | | | |
| ester; Ramipril | | | |
| Isopropyl Ester | | | |
| and | | | |
| Hexahydroramipril | | | |
| Initial | ND | ND | ND |
| 2 weeks | ND | ND | ND |

ND = None Detected
Conditions tested 40° C./75% RH

Long-Term Stability of Ramipril Tablets

DKP rate up to about 36 months is shown in FIGS. 11A-11C. DKP formation is less than about 0.05% after 3 months and less than an extrapolated amount of about 3.0% after about 36 months in the examples tested. In addition to DKP formation other degradation pathways for ramipril exist, including formation of ramiprilat (ramipril diacid). Premature formation (before patient administration) of ramiprilat is undesirable because it is not absorbed by the patient, and is therefore insufficiently bioavailable. Preferably, stability analyses should include detection of levels of ramiprilat.

While the present invention has been described in the context of numerous embodiments and examples, it will be readily apparent to those skilled in the art that other modifications and variations can be made therein without departing from the spirit or scope of the present invention. Accordingly, it is not intended that the present invention be limited to the specifics of the foregoing description of the exemplary embodiments and example.

1. A pharmaceutical composition comprising ramipril coated by a blending agent, wherein the blending agent is selected from; glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

2. The composition of claim 1, wherein the blending agent is glyceryl behenate.

3. The composition of claim 1, wherein about 50 to 100% of the ramipril is coated by the blending agent.

4. The composition of claim 1, wherein about 75 to 100% of the ramipril is coated by the blending agent.

5. The composition of claim 1, wherein about 95 to 100% of the ramipril is coated by the blending agent.

6. The composition of claim 1, wherein the blending agent is at least 0.1% by weight.

7. The composition of claim 1, wherein the blending agent is at least 1% by weight.

8. The composition of claim 1, wherein the blending agent is at least 4% by weight.

9. The composition of claim 1, wherein the ramipril is substantially stable against decomposition into a degradant product.

10. The composition of claim 9, wherein the degradant product is ramipril-diacid or ramipril-diketopiperazine.

11. The composition of claim 10, wherein the rate of decomposition of the ramipril to ramipril-diketopiperazine is less than about 0.3% by weight during about the first three months.

12. The composition of claim 10, wherein the rate of decomposition of the ramipril to ramipril-diketopiperazine is less than about 3.0% by weight during about the first thirty-six months.

13. The composition of claim 10, wherein the rate of decomposition of the ramipril to ramipril-diketopiperazine is less than about 0.09% by weight, on average, per month.

14. The composition of claim 1, wherein the ramipril is coated ramipril.

15. The composition of claim 1, wherein the composition is a solid dosage form.

16. The composition of claim 1, wherein the composition is an oral dosage form.

17. The composition of claim 1, wherein the composition is a tablet, caplet or capsule.

18. The composition of claim 17, wherein the composition is a tablet.

19. The composition of claim 1, wherein the composition further comprises an excipient.

20. The composition of claim 1, wherein the ramipril is between the amount of about 0.1 mg to 50 mg.

21. The composition of claim 1, wherein the ramipril is between the amount of about 1.25 mg to 25 mg.

22. The composition of claim 1, wherein the ramipril is between the amount of about 10 mg to 20 mg.

23. The composition of claim 1, wherein the ramipril is between the amount of about 10 or 20 mg.

24. A pharmaceutical composition comprising ramipril, wherein the ramipril is coated by a blending agent, wherein the rate of decomposition of the ramipril to ramipril-diketopiperazine is less than about 0.4% of the total weight of ramipril during the first 3 months when the pharmaceutical composition is at room temperature.

25. The composition of claim 24, wherein the rate of decomposition is about 0.3% of the total weight of ramipril during the first 3 months when the pharmaceutical composition is at room temperature.

26. The composition of claim 23, wherein the composition is a solid dosage form.

27. The composition of claim 23, wherein the composition is an oral dosage form.

28. The composition of claim 23, wherein the composition is a tablet, caplet or capsule.

29. The composition of claim 29, wherein the composition is a tablet.

30. The composition of claim 23, wherein the ramipril is between the amount of about 1.25 mg to 25 mg.

31. The composition of claim 23, wherein the ramipril is between the amount of about 10 mg to 20 mg.

32. The composition of claim 23, wherein the ramipril is in the amount of about 10 mg or 20 mg.

33. The composition of claim 23, wherein the ramipril is coated ramipril.

34. A pharmaceutical composition comprising ramipril, wherein the ramipril is coated by a blending agent, wherein the rate of decomposition of the ramipril to ramipril-diketopiperazine is less than about 0.75% of the total weight of ramipril during the first 6 months when the pharmaceutical composition is at room temperature.

35. The composition of claim 34, wherein the rate of decomposition is about 5% of the total weight of ramipril during the first 6 months when the pharmaceutical composition is at room temperature.

36. The composition of claim 34, wherein the composition is a solid dosage form.

37. The composition of claim 34, wherein the composition is an oral dosage form.

38. The composition of claim 34, wherein the composition is a tablet, caplet or capsule.

39. The composition of claim 39, wherein the composition is a tablet.

40. The composition of claim 34, wherein the ramipril is between the amount of about 1.25 mg to 25 mg.

41. The composition of claim 34, wherein the ramipril is in the amount of about 10 mg to 20 mg.

42. The composition of claim 34, wherein the ramipril is in the amount of about 10 or 20 mg.

43. The composition of claim 34, wherein the ramipril is coated ramipril.

44. A pharmaceutical composition comprising ramipril, wherein the ramipril is coated by a blending agent, wherein the rate of decomposition of the ramipril to ramipril-diketopiperazine is less than about 3.0% of the total weight of ramipril during the first 36 months when the pharmaceutical composition is at room temperature.

45. The composition of claim 44, wherein the rate of decomposition is about 2.0% of the total weight of ramipril during the first 36 months when the pharmaceutical composition is at room temperature.

46. The composition of claim 44, wherein the rate of decomposition is about 1.5% of the total weight of ramipril during the first 36 months when the pharmaceutical composition is at room temperature.

47. The composition of claim 44, wherein the composition is a solid dosage form.

48. The composition of claim 44, wherein the composition is an oral dosage form.

49. The composition of claim 44, wherein the composition is a tablet, caplet or capsule.

50. The composition of claim 49, wherein the composition is a tablet.

51. The composition of claim 44, wherein the ramipril is between the amount of about 1.25 mg to 25 mg.

52. The composition of claim 44, wherein the ramipril is between the amount of about 10 mg to 20 mg.

53. The composition of claim 44, wherein the ramipril is between the amount of about 10 mg or 20 mg.

54. The composition of claim 44, wherein the coated ramipril is coated ramipril particles.

55. A pharmaceutical composition comprising ramipril, wherein the ramipril is coated by a blending agent, wherein the rate of decomposition of the ramipril to ramipril-diketopiperazine is less than about 0.09%, on average, of the total weight of ramipril per month when the pharmaceutical composition is at room temperature.

56. The composition of claim 55, wherein the rate of decomposition is about 0.05% or less on average, of the total weight of ramipril per month when the pharmaceutical composition is at room temperature.

57. The composition of claim 55, wherein the composition is a solid dosage form.

58. The composition of claim 55, wherein the composition is an oral dosage form.

59. The composition of claim 55, wherein the composition is a tablet, caplet or capsule.

60. The composition of claim 59, wherein the composition is a tablet.

61. The composition of claim 55, wherein the ramipril is in the amount of about 1.25 mg to 25 mg.

62. The composition of claim 55, wherein the ramipril is between the amount of about 10 mg to 20 mg.

63. The composition of claim 55, wherein the ramipril is between the amount of about 10 or 20 mg.

64. The composition of claim 55, wherein the ramipril is coated ramipril.

65. A method of making a pharmaceutical composition comprising combining ramipril with a blending agent, wherein the ramipril is coated by blending agent.

66. The composition of claim 65, wherein about 50 to 100% of the ramipril is coated by the blending agent.

67. The composition of claim 65, wherein about 75 to 100% of the ramipril is coated by the blending agent.

68. The composition of claim 65, wherein about 95 to 100% of the ramipril is coated by the blending agent.

69. A method of making a pharmaceutical composition comprising first pre-blending or co-milling ramipril with a blending agent, wherein the blending agent is selected from; glyceryl behenate, glyceryl stearate, stearyl alcohol, macrogol stearate ether, palmitostearate, ethylene glycol, polyethylene glycol, stearic acid, cetyl alcohol, lauryl alcohol, amylopectin, poloxymer or combinations thereof.

70. The method of claim 69, further comprising adding a diluent, lubricant, disintegrant or a combination thereof.

71. The method of claim 69, further comprising compressing the ramipril with a blending agent into tablets.

72. The method of claim 69, wherein the blending agent is glyceryl behenate.

73. The method of claim 69, wherein the blending agent is at least 0.1% by weight.

74. The method of claim 69, wherein the blending agent is at least 1% by weight.

75. The method of claim 69, wherein the blending agent is at least 4% by weight.

76. The method of claim 69, wherein the ramipril is coated ramipril.

77. The method of claim 69, wherein the composition is a solid dosage form.

78. The method of claim 69, wherein the composition is an oral dosage form.

79. The method of claim 69, wherein the composition is a tablet, caplet or capsule.

80. The method of claim 79, wherein the composition is a tablet.

81. The method of claim 69, wherein the ramipril is in the amount of about 0.1 mg to 50 mg.

82. The method of claim 69, wherein the ramipril is in the amount of about 1.25 mg to 25 mg.

83. The method of claim 69, wherein the ramipril is in the amount of about 10 mg to 20 mg.

84. The method of claim 69, wherein the ramipril is in the amount of about 10 mg or 20 mg.

85. A method of making a pharmaceutical composition comprising first pre-blending and/or co-milling ramipril and glyceryl behenate; and combining the ramipril and glyceryl behenate with microcrystalline cellulose and croscarmellose sodium.

86. A product made by the process of claim 85.

87. A method of treating a cardiovascular disorders comprising administering a composition as claimed in claims 1.

88. A method of treating the cardiovascular disorder of claim 87, wherein the cardiovascular disorder is hypertension, heart failure, congestive heart failure, myocardial infarction, atherosclerotic cardiovascular disease, asymptomatic left ventricular dysfunction, chronic renal insufficiency, and diabetic or hypertensive nephropathy.

89. A stable pharmaceutical composition comprising: an intimate admixture including a 2-aza-bicyclo[3.3.0]-octane-3-carboxylic acid derivative and an effective amount of a lubricant to stabilize the composition; and an external excipient.

90. The composition according to claim 89, wherein the intimate admixture is in granular form.

91. The composition according to claim 89, wherein the effective amount of the lubricant ranges from about 0.3% to about 60% by weight of the intimate admixture.

92. The composition according to claim 89, wherein the effective amount of the lubricant ranges from about 0.8% to about 50% by weight of the intimate admixture.

93. The composition according to claim 89, wherein the effective amount of the lubricant ranges from about 1% to about 40% by weight of the intimate admixture.

94. The composition according to claim 89, wherein the effective amount of the lubricant ranges from about 2% to about 10% by weight of the intimate admixture.

95. The composition according to claim 89, wherein the lubricant is selected from the group consisting of magnesium stearate, talc, stearic acid, glycerylbehenate, polyethylene glycol, ethylene oxide polymers, sodium lauryl sulfate, magnesium lauryl sulfate, sodium oleate, DL-leucine, and sodium stearyl fumarate.

96. The composition according to claim 95, wherein the lubricant is sodium stearyl fumarate.

97. The composition according to claim 89, wherein the intimate admixture further comprises one non-lubricant excipient.

98. The composition according to claim 97, wherein the non-lubricant excipient is microcrystalline cellulose.

99. The composition according to claim 97, wherein the non-lubricant excipient is in the amount of about 95% or less by weight of the intimate admixture.

100. The composition according to claim 89, further comprising a diuretic agent.

101. The composition according to claim 100, wherein the diruetic agent is hydrochlorothiazide.

102. The composition according to claim 89, wherein the derivative is selected from the group consisting of ramipril, quinapril, moexipril, fosinopril, enalapril, perindopril, and trandolapril.

103. The composition according to claim 102, wherein the derivative is ramipril.

104. The composition according to claim 89, wherein the derivative is present in an amount of from about 0.3% to about 6% by weight of the total composition.

105. The composition according to claim 89, wherein the derivative is present in an amount of from about 0.8% to about 5% by weight of the total composition.

106. The composition according to claim 89, wherein the derivative is present in an amount of from about 0.8% to about 4.2% by weight of the total composition.

107. The composition according to claim 89, wherein the composition is in solid unit dosage form.

108. The composition according to claim 107, wherein the composition is in tablet form.

109. The composition according to claim 107, wherein the composition is in capsule form.

110. The composition according to claim 89, wherein the amount of a principal degradant present in the composition after 48 hours at 55° C. is less than 3% by weight of the derivative.

111. The composition according to claim 89, wherein the amount of a principal degradant present in the composition after 48 hours at 55° C. is less than 1% by weight of the derivative.

112. The composition according to claim 111, wherein the principal degradant is diketopiperazine and the derivative is rampiril.

113. The composition according to claim 112, wherein the principal degradant is diketopiperazine and the derivative is rampiril.

114. The composition according to claim 89, wherein the amount of an active form degradant present in the composition after 48 hours at 55° C. is less than 0.3% by weight of the derivative.

115. The composition according to claim 89, wherein the amount of an active form degradant present in the composition after 48 hours at 55° C. is less than 0.2% by weight of the derivative.

116. The composition according to claim 89, wherein the total amount of an active form degradant and a principal degradant present in the composition after 48 hours at 55° C. is less than 3.3% by weight of the derivative.

117. The composition according to claim 89, wherein the total amount of an active form degradant and a principal degradant present in the composition after 48 hours at 55° C. is less than 1% by weight of the derivative.

118. The composition according to claim 117, wherein the active form degradant is ramprilat, the principal degradant is diketopiperazine, and the derivative is rampiril.

119. The composition according to claim 118, wherein the active form degradant is ramprilat, the principal degradant is diketopiperazine, and the derivative is rampiril.

120. A method for preparing a stable pharmaceutical composition comprising:
forming an intimate admixture including a 2-aza-bicyclo[3.3.0]-octane-3-c-arboxylic acid derivative and a lubricant; and blending the intimate admixture with an external excipient.

121. The method of claim 120, further comprising transforming the final blend into solid unit dosage form.

122. The method of claim 121, wherein the composition is in tablet form.

123. The method of claim 121, wherein the composition is in capsule form.

124. The method of claim 123, wherein the intimate admixture is in granular form.

125. The method of claim 123, wherein the intimate admixture is formed by dry granulation or wet granulation.

126. A 2-aza-bicyclo[3.3.0]-octane-3-carboxylic acid derivative composition made by the process of claim 32.
